(19) United States
(12) Patent Application Publication
(10) Pub. No.: US 2008/0280766 A1
(43) Pub. Date: Nov. 13, 2008

(54) Pyridopyrazines for Combating Phytopathogenic Fungi

(75) Inventors: Patrick Jelf Crowley, Berkshire (GB); Urs Muller, Basel (CH); Markus Dobler, Cambridge, MA (US); John Williams, Berkshire (GB)

Correspondence Address: SYNGENTA CROP PROTECTION , INC.;PATENT AND TRADEMARK DEPARTMENT, 410 SWING ROAD, GREENSBORO, NC, 27409, US

(73) Assignees: SYNGENTA CROP PROTECTION, INC., Greensboro, NC (US); SYNGENTA LIMTED, Guildford, Surrey (GB)

(21) Appl. No.: 11/570,984

(22) PCT Filed: Jun. 21, 2005
(86) PCT No.: PCT/EP05/06706

§ 371 Date: Dec. 20, 2006

(30) Foreign Application Priority Data
Jun. 22, 2004 (GB) 0413953.1

(51) Int. Cl. A01N 43/90; C07D 471/04; A01P 3/00

(52) U.S. Cl. 504/235; 544/350

(57) ABSTRACT

The compounds of the general formula (I) wherein R, R¹, R², R⁸, and R⁹ are defined as set forth in the specification.

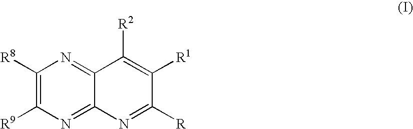

This invention relates to novel derivatives of pyridopyrazines, to processes for preparing them, to certain intermediate chemicals used in their manufacture, to compositions containing them and to methods of using them to combat fungi, especially fungal infections of plants.

Derivatives of the nitrogen-containing 5,6 ring system s-1,2,4-triazolo[1,5-a]pyrimidine are known from the patent literature as being useful for controlling phytopathogenic fungi. Examples of recent patent publications include EP-A-1249452, WO 02/051845, WO 02/083676, WO 02/083677, WO 02/088125, WO 02/088126, WO 02/088127. Derivatives of pyridopyrazines are known in the chemical literature, for example from J. Med. Chem. (1968), 11(6), 1216-18, J. Med. Chem. (1970), 13(5), 853-7 and U.S. Pat. No. 3,984,412, but not for agrochemical purposes.

The present invention is concerned with the provision of novel pyridopyrazines, for combating phytopathogenic diseases on plants and harvested food crops.

Thus, according to the present invention, there is provided a compound of the general formula (I):

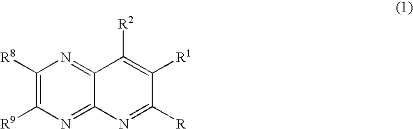

wherein
R is H, halo, C₁₋₄ alkyl, C₁₋₄ alkoxy, halo(C₁₋₄)alkyl, cyano or NR³R⁴;
R¹ is aryl, aryloxy, arylthio, heteroaryl, heteroaryloxy, heteroarylthio, aryl(C₁₋₄)alkyl, aryl(C₁₋₄)alkoxy, heteroaryl(C₁₋₄)alkyl, heteroaryl(C₁₋₄)alkoxy;
R² is halo or NR³R⁴;
R³ and R⁴ are independently H, C₁₋₈ alkyl, C₂₋₈ alkenyl, C₂₋₈ alkynyl, aryl, aryl(C₁₋₈)alkyl, C₃₋₈ cycloalkyl, C₃₋₈cycloalkyl(C₁₋₆)alkyl, heteroaryl, heteroaryl(C₁₋₈)alkyl, NR⁵R⁵, provided that not both R³ and R⁴ are H or NR⁵R⁶, or
R³ and R⁴ together form a C₃₋₇ alkylene or C₃₋₇ alkenylene chain optionally substituted with one or more C₁₋₄alkyl or C₁₋₄ alkoxy groups, or, together with the nitrogen atom to which they are attached, R³ and R⁴ form a morpholine, thiomorpholine, thiomorpholine S-oxide, or thiomorpholine S-dioxide ring, or a piperazine or piperazine N—(C₁₋₄)alkyl (especially N-methyl) ring or a pyrrolidine ring;
R⁵ and R⁶ are independently H, C₁₋₈ alkyl, C₂₋₈ alkenyl, C₂₋₈ alkynyl, aryl, aryl(C₁₋₈)alkyl, C₃₋₈ cycloalkyl, C₃₋₈cycloalkyl(C₁₋₆)alkyl, heteroaryl or heteroaryl(C₁₋₈)alkyl;
R⁸ and R⁹ can be H, halo, C₁₋₄alkyl, C₁₋₄alkoxy, halo(C₁₋₄)alkyl, CN, C₁₋₄alkylthio, C₁₋₄alkylsulphinyl, C₁₋₄alkylsulphonyl, aryl, heteroaryl, halo(C₁₋₆)alkoxy, halo(C₁₋₄)alkylthio, C₂₋₄alkenyl, C₂₋₆alkynyl, C₃₋₆cycloalkyl, C₁₋₆alkoxycarbonyl, R³R⁴NCO, NR¹⁰R¹¹, or R⁸ and R⁹ can be joined to form a saturated or unsaturated 5-7 membered carbocyclic or heterocyclic ring, optionally substituted with one or two substituents R¹² and where the heterocyclic ring can contain from one to three heteroatoms chosen from NR¹³, O or S;
R¹⁰ and R¹¹ are independently H, C₁₋₈ alkyl, C₂₋₈alkenyl, C₂₋₈ alkynyl, C₁₋₄ alkoxy, aryl, aryl(C₁₋₈)alkyl, C₃₋₈cycloalkyl, C₃₋₈cycloalkyl(C₁₋₁₆)alkyl, heteroaryl, heteroaryl(C₁₋₈)alkyl, NR⁵R⁶, provided that not both R⁹ and R¹⁰ are NR⁵R⁶ or C₁₋₄alkoxy;
R¹² is H, halo, C₁₋₄alkyl, C₁₋₄alkoxy, halo(C₁₋₄)alkyl, CN, C₁₋₄alkylthio, C₁₋₄alkylsulphinyl, C₁₋₄alkylsulphonyl, aryl, heteroaryl, halo(C₁₋₆)alkoxy, halo(C₁₋₄)alkylthio, C₂₋₄alkenyl, C₂₋₆alkynyl, C₃₋₆cycloalkyl, NR¹⁰R¹¹, aryl, heteroaryl, halo(C₁₋₆)alkoxy, halo(C₁₋₄)alkylthio, C₂₋₄alkenyl, C₂₋₆alkynyl, C₃₋₆cycloalkyl, C₁₋₆alkoxycarbonyl or R³R⁴NCO;
R¹³ is H, C₁₋₄alkyl or halo(C₁₋₄)alkyl; any of the foregoing alkyl, alkenyl, alkynyl or cycloalkyl groups or moieties being optionally substituted with halogen, cyano, C₁₋₆ alkoxy, C₁₋₆ alkylcarbonyl, C₁₋₆ alkoxycarbonyl, C₁₋₆ haloalkoxy, C₁₋₆ alkylthio, tri(C₁₋₄)alkylsilyl, C₁₋₆ alkylamino or C₁₋₆ dialkylamino, any of the foregoing morpholine, thiomorpholine, piperidine, piperazine and pyrrolidine rings being optionally substituted with C₁₋₄ alkyl (especially methyl), and any of the foregoing aryl or heteroaryl groups or moieties being optionally substituted with one or more substituents selected from halo, hydroxy, mercapto, C₁₋₆ alkyl, C₂₋₆ alkenyl, C₂₋₆ alkynyl, C₁₋₆ alkoxy, C₂₋₆ alkenyloxy, C₂₋₆ alkynyloxy, halo(C₁₋₁₆)alkyl, halo(C₁₋₆)alkoxy, C₁₋₆ alkylthio, halo(C₁₋₆)alkylthio, hydroxy(C₁₋₆)alkyl, C₁₋₄alkoxy(C₁₋₆)alkyl, C₃₋₆ cycloalkyl, C₃₋₆ cycloalkyl(C₁₋₄)alkyl, aryl, heteroaryl, aryloxy, heteroaryloxy, aryl(C₁₋₄)alkoxy, heteroaryl(C₁₋₄)alkoxy, aroyloxy, arylthio, arylsulphinyl, arylsulphonyl, heteroarylthio, heteroarylsulphinyl, heteroarylsulphonyl, arylalkenyl, arylalkynyl, heteroarylalkenyl, heteroarylalkynyl, aryl(C₁₋₄)alkyl, heteroaryl(C₁₋₄)alkyl, cyano, isocyano, thiocyanato, isothiocyanato, nitro, —NR¹⁰³R¹⁰⁴, —NHCOR¹⁰³—NHCONR¹⁰³R¹⁰⁴, —CONR¹⁰³R¹⁰⁴, —SO₂R¹⁰³, —SO₂R¹⁰³—COR¹⁰³—CR¹⁰³═NR¹⁰⁴ or —N═CR¹⁰³R¹⁰⁴, in which R¹⁰³ and R¹⁰⁴ are independently hydrogen, C₁₋₄ alkyl, halo-(C₁₋₄)alkyl, C₁₋₄ alkoxy, halo(C₁₋₄)alkoxy, C₁₋₄ alkylthio, C₃₋₆ cycloalkyl, C₃₋₆ cycloalkyl(C₁₋₄)-alkyl, phenyl or benzyl, with any of the forgoing aryl or heteroaryl substituents being optionally substituted with halo, C₁₋₆ alkyl, C₂₋₆ alkenyl, C₂₋₆ alkynyl, C₁₋₆ alkoxy, C₂₋₆ alkenyloxy, C₂₋₆ alkynyloxy, halo(C₁₋₆)alkyl, halo(C₁₋₈)alkoxy, C₁₋₆ alkylthio, halo(C₁₋₆)-alkylthio, C₁₋₄ alkoxy(C₁₋₆)alkyl, C₃₋₆ cycloalkyl, cyano or nitro, provided that not both of R⁸ and R⁹ are independently hydrogen, halogen, C₁₋₄alkyl, C₁₋₄alkoxy, haloC₁₋₄alkyl.

The compounds of the invention may contain one or more asymmetric carbon atoms and may exist as enantiomers (or as pairs of diastereoisomers) or as mixtures of such. They may also exist as diastereoisomers by virtue of restricted rotation about a bond. However, mixtures of enantiomers or diastereoisomers may be separated into individual isomers or isomer pairs, and this invention embraces such isomers and mixtures thereof in all proportions. It is to be expected that for any given compound, one isomer may be more fungicidally active than another.

Except where otherwise stated, alkyl groups and alkyl moieties of alkoxy, alkylthio, etc., contain from 1 to 8, suitably from 1 to 6 and typically from 1 to 4, carbon atoms in the form of straight or branched chains. Examples are methyl, ethyl, n- and isopropyl, n-, sec-, iso- and tert-butyl, n-pentyl and n-hexyl. Cycloalkyl groups contain from 3 to 8, typically from 3 to 6, carbon atoms and include bicycloalkyl groups such as the bicyclo[2.2.1]heptyl group. Haloalkyl groups or moieties are typically trichloromethyl or trifluoromethyl or contain a trichloromethyl or trifluoromethyl terminal group.

Except where otherwise stated, alkenyl and alkynyl moieties also contain from 2 to 8, suitably from 2 to 6 and typically from 2 to 4, carbon atoms in the form of straight or branched chains. Examples are allyl, 2-methylallyl and propargyl. Optional substituents include halo, typically fluoro. An example of halo-substituted alkenyl is 3,4,4-trifluoro-n-butenyl.

Halo includes fluoro, chloro, bromo and iodo. Most commonly it is fluoro, chloro or bromo and usually fluoro or chloro.

Aryl is usually phenyl but also includes naphthyl, anthryl and phenanthryl, biphenyl.

Heteroaryl is typically a 5- or 6-membered aromatic ring containing one or more O, N or S heteroatoms, which may be fused to one or more other aromatic or heteroaromatic rings, such as a benzene ring. Examples are thienyl, furyl, pyrrolyl, isoxazolyl, oxazolyl, oxadiazolyl, pyrazolyl, imidazolyl, triazolyl, isothiazolyl, tetrazolyl, thiadiazolyl, pyridyl, pyrimidinyl, pyrazinyl, pyridazinyl, triazinyl, benzofuryl, benzothienyl, dibenzofuryl, benzothiazolyl, benzoxazolyl, benzimidazolyl, indolyl, quinolinyl and quinoxalinyl groups and, where appropriate, N-oxides thereof.

Where R⁸ and R⁹ can be joined to form a saturated or unsaturated 5-7 membered carbocyclic or heterocyclic ring, examples of such rings are shown below:

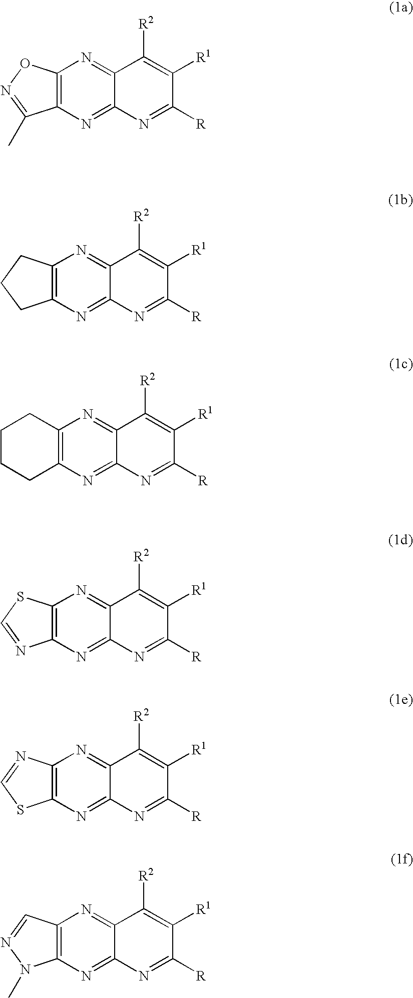

Typically, the compound of formula (1) is a compound of formula (1g).

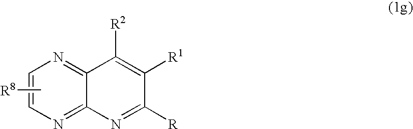

One of R and R², preferably R², is NR³R⁴. The other is typically halo, especially chloro or fluoro. The more active compounds are those where R² is NR³R⁴. R³ is typically C₁₋₈ alkyl (for example ethyl, n-propyl, n-butyl, sec-butyl (the S- or R-isomer or the racemate), isobutyl and tert-butyl), halo(C₁₋₈)alkyl (for example 2,2,2-trifluoroethyl, 2,2,2-trifluoro-1-methylethyl (the S- or R-isomer or the racemate), 2,2,2-trifluoro-1-methylpropyl (the S- or R-isomer or the racemate), 3,3,3-trifluoropropyl and 4,4,4-trifluorobutyl), C₁₋₄ alkoxy(C₁₋₈)alkyl (for example methoxymethyl and methoxy-isobutyl), C₁₋₄ alkoxyhalo(C₁₋₈)-alkyl (for example 2-methoxy-2-trifluoromethylethyl), C₁₋₄ alkylcarbonyl(C₁₋₈)alkyl (for example 1-acetylethyl and 1-tert-butylcarbonylethyl), C₁₋₄ alkylcarbonylhalo(C₁₋₈)alkyl (for example 1-acetyl-2,2,2-trifluoroethyl), phenyl(₁₋₄)alkyl (for example benzyl), C₂₋₈ alkenyl (for example allyl and methylallyl), halo(C₂₋₈)alkenyl (for example 3-methyl-4,4-difluorobut-3-enyl), C₂₋₈ alkynyl (for example propargyl), C₃₋₈ cycloalkyl (for example cyclopropyl, cyclobutyl, cyclopentyl and cyclohexyl) optionally substituted with chloro, fluoro or methyl, C₃₋₈ cycloalkyl(C₁₋₄)alkyl (for example cyclopropylmethyl, cyclobutylmethyl, cyclopentylmethyl and cyclohexylmethyl), phenylamino, piperidino or morpholino, the phenyl ring of phenylalkyl or phenylamino being optionally substituted with one, two or three substituents selected from halo (typically fluoro, chloro or bromo), C₁₋₄ alkyl (typically methyl), halo(C₁₋₄)alkyl (typically trifluoromethyl), C₁₋₄ alkoxy (typically methoxy) and halo(C₁₋₄)alkoxy (typically trifluoromethoxy). R⁴ is typically H, C₁₋₄ alkyl (for example ethyl and n-propyl), halo(C₁₋₄)alkyl (for example 2,2,2-trifluoroethyl) or amino. Alternatively R³ and R⁴ together form a C₄₋₆ alkylene chain optionally substituted with methyl, for example 3-methylpentylene, or, together with the nitrogen atom to which they are attached, R³ and R⁴ form a morpholine, piperidine, thiomorpholine, thiomorpholine S-oxide, or thiomorpholine S-dioxide ring, or a piperazine or piperazine N—(C₁₋₄)alkyl (especially N-methyl) ring or a pyrrolidine ring, in which the morpholine, piperidine, piperazine or pyrrolidine rings are optionally substituted with methyl.

Typically R¹ is phenyl optionally substituted with from one to five halogen atoms, particularly fluorine and chlorine atoms and especially fluorine atoms or with from one to three substituents selected from halo (for example fluoro and chloro), C₁₋₄ alkyl (for example methyl), halo(C₁₋₄)alkyl (for example trifluoromethyl), C₁₋₄ alkoxy (for example methoxy) or halo(C₁₋₄)alkoxy (for example trifluoromethoxy). Examples are 2,6-difluorophenyl, 2-fluoro-6-chlorophenyl, 2,5,6-trifluorophenyl, 2,4,6-trifluorophenyl, 2,6-difluoro-4-methoxyphenyl, pentafluorophenyl, 2-fluorophenyl, 2,3,5,6-tetrafluorophenyl, 2-chloro-4,6-difluorophenyl, 2-chlorophenyl, 2,6-dichlorophenyl, 2,4-dichlorophenyl, 2,4,6-trichlorophenyl, 2,3,6-trichlorophenyl, pentachlorophenyl, 2-fluoro-4,6-dichlorophenyl, 4-fluoro-2,6-dichlorophenyl, 2-bromophenyl, 2-fluoro-6-bromophenyl, 2-bromo-4,6-difluorophenyl, 2-fluoro-6-methylphenyl, 2-chloro-6-methylphenyl, 2-methoxyphenyl, 2,6-dimethoxyphenyl, 2-fluoro-6-methoxyphenyl, 2-trifluoromethylphenyl, 2-fluoro-6-trifluoromethylphenyl, 2,6-di-(trifluoromethyl)phenyl, 2-chloro-6-trifluoromethylphenyl, 2,4-difluoro-6-trifluoromethylphenyl, 2,4-difluoro-6-methoxyphenyl and 2,4-difluoro-6-methylphenyl.

Also of particular interest are compounds where R¹ is pyridyl optionally substituted with from one to four halogen atoms or with from one to three substituents selected from halo (for example fluoro and chloro), C₁₋₄ alkyl (for example methyl), halo(C₁₋₄)alkyl (for example trifluoromethyl), C₁₋₄ alkoxy (for example methoxy) or halo(C₁₋₄)alkoxy (for example trifluoromethoxy). Examples are 2,4-difluoropyrid-3-yl, 3,5-difluoropyrid-4-yl, tetrafluoropyrid-4-yl, 3-fluoropyrid-2-yl, 4-fluoropyrid-3-yl, 3-fluoropyrid-4-yl, 2-fluoropyrid-3-yl, 2,4,6-trifluoropyrid-3-yl, 3,5-difluoropyrid-2-yl, 2,6-difluoropyrid-3-yl, 2,4-difluoro-6-methoxypyrid-3-yl, 2-fluoro-4-chloropyrid-3-yl, 3-fluoro-5-chloropyrid-4-yl, 2-chloro-4-fluoropyrid-3-yl, 2,4-dichloropyrid-3-yl, 3-chloropyrid-2-yl, 1,4-chloropyrid-3-yl, 3-chloropyrid-4-yl, 2-chloropyrid-3-yl, 3-trifluoromethylpyrid-2-yl, 4-trifluoromethylpyrid-3-yl, 3,5-dichloropyrid-2-yl, 4,6-dichloropyrid-3-yl, 3-trifluoromethylpyrid-4-yl, 2-trifluoromethylpyrid-3-yl, 2-fluoro-4-trifluoromethylpyrid-3-yl, 3-fluoro-5-trifluoromethylpyrid-4-yl, 4-fluoro-2-trifluoromethylpyrid-3-yl, 2,6-dichloropyrid-3-yl, 3,5-dichloropyrid-4-yl, 3-chloro-5-trifluoromethylpyrid-2-yl, 3-fluoro-6-trifluoromethylpyrid-2-yl, pyrid-2-yl, pyrid-3-yl and pyrid-4-yl.

In one aspect the invention provides a compound of the general formula (1) wherein R⁸ and R⁹ can be H, halo, C₁₋₄alkyl, C₁₋₄alkoxy, halo(C₁₋₄)alkyl, CN, C₁₋₄alkylthio, C₁₋₄alkylsulphinyl, C₁₋₄alkylsulphonyl, aryl, heteroaryl, halo(C₁₋₆)alkoxy, halo(C₁₋₄)alkylthio, C₂₋₄alkenyl, C₂₋₆alkynyl, C₃₋₆cycloalkyl, C₁₋₆alkoxycarbonyl, R³R⁴NCO, NR¹⁰R¹¹, or R³ and R⁹ can be joined to form a saturated or unsaturated 5-7 membered carbocyclic or heterocyclic ring, optionally substituted with one or two substituents R¹² where the heterocyclic ring can contain from one to three heteroatoms chosen from NR¹³, O or S; one of R and R² (preferably R²) is NR³R⁴ and the other is halo;

R¹ is aryl, aryloxy, arylthio, heteroaryl, heteroaryloxy, heteroarylthio, aryl(C₁₋₄)alkyl, aryl(C₁₋₄)alkoxy, heteroaryl(C₁₋₄)alkyl;
R³ and R⁴ are independently H, C₁₋₈ alkyl, C₂₋₈ alkenyl, C₂₋₈ alkynyl, aryl, aryl(C₁₋₈)alkyl, C₃₋₈ cycloalkyl, C₃₋₈ cycloalkyl(C₁₋₆)alkyl, heteroaryl, heteroaryl(C₁₋₈)alkyl, NR⁵R⁶, provided that not both R³ and R⁴ are H or NR⁵R⁶, or
R³ and R⁴ together form a C₃₋₇ alkylene or C₃₋₇ alkenylene chain optionally substituted with one or more C₁₋₄ alkyl or C₁₋₄ alkoxy groups, or,
together with the nitrogen atom to which they are attached, R³ and R⁴ form a morpholine, thiomorpholine, thiomorpholine S-oxide or thiomorpholine S-dioxide ring or a piperidine, piperazine or piperazine N—(C₁₋₄)alkyl (especially N-methyl) ring; and
R⁵ and R⁶ are independently H, C₁₋₈ alkyl, C₂₋₈ alkenyl, C₂₋₈ alkynyl, aryl, aryl(C₁₋₈)alkyl, C₃₋₈ cycloalkyl, C₃₋₈ cycloalkyl(C₁₋₆)alkyl, heteroaryl or heteroaryl(C₁₋₈)alkyl; any of the foregoing alkyl, alkenyl, alkynyl or cycloalkyl groups or moieties being optionally substituted with halogen, cyano, C₁₋₆ alkoxy, C₁₋₁₆ alkylcarbonyl, C₁₋₆ alkoxycarbonyl, C₁₋₆ haloalkoxy, C₁₋₆ alkylthio, tri(C₁₋₄)alkylsilyl, C₁₋₆ alkylamino or C₁₋₆ dialkylamino, any of the foregoing morpholine, thiomorpholine, piperidine, piperazine and pyrrolidine rings being optionally substituted with C₁₋₄ alkyl (especially methyl), and any of the foregoing aryl, heteroaryl, aryloxy or heteroaryl groups being optionally substituted with one or more substituents selected from halo, hydroxy, mercapto, C₁₋₆ alkyl, C₂₋₆ alkenyl, C₂₋₆ alkynyl, C₁₋₆ alkoxy, C₂₋₆ alkenyloxy, C₂₋₆ alkynyloxy, halo(C₁₋₆)alkyl, halo(C₁₋₆)alkoxy, C₁₋₆ alkylthio, halo(C₁₋₆)alkylthio, hydroxy(C₁₋₆)alkyl, C₁₋₄alkoxy(C₁₋₆)alkyl, C₃₋₆ cycloalkyl, C₃₋₆ cycloalkyl(C₁₋₄)alkyl, aryl, heteroaryl, aryloxy, heteroaryloxy, aryl(C₁₋₄)alkoxy, heteroaryl(C₁₋₄)alkoxy, aroyloxy, arylthio, arylsulphinyl, arylsulphonyl, heteroarylthio, heteroarylsulphinyl, heteroarylsulphonyl, arylalkenyl, arylalkynyl, heteroarylalkenyl, heteroarylalkynyl, aryl(C₁₋₄)alkyl, heteroaryl(C₁₋₄)alkyl, cyano, isocyano, thiocyanato, isothiocyanato, nitro, —NR¹⁰³R¹⁰⁴, —NHCOR¹⁰³, —NHCONR¹⁰³R¹⁰⁴, —CONR¹⁰³R¹⁰⁴, —SO₂R¹⁰³, —OSO₂R¹⁰³, —COR¹⁰³, —R¹⁰³═NR¹⁰⁴ or —N═CR¹⁰³R¹⁰⁴, in which R¹⁰³ and R¹⁰⁴ are independently hydrogen, C₁₋₄ alkyl, halo(C₁₋₄)alkyl, C₁₋₄ alkoxy, halo(C₁₋₄)alkoxy, C₁₋₄ alkylthio, C₃₋₆ cycloalkyl, C₃₋₆ cycloalkyl(C₁₋₄)alkyl, phenyl or benzyl, with any of the forgoing aryl or heteroaryl substituents being optionally substituted with halo, C₁₋₆ alkyl, C₂₋₆ alkenyl, C₂₋₆ alkynyl, C₁₋₆ alkoxy, C₂₋₆ alkenyloxy, C₂₋₆ alkynyloxy, halo(C₁₋₆)alkyl, halo(C₁₋₆)alkoxy, C₁₋₆ alkylthio, halo(C₁₋₆)alkylthio, C₁₋₄ alkoxy(C₁₋₆)alkyl, C₃₋₆ cycloalkyl, cyano or nitro, provided that not both of R⁸ and R⁹ are independently H, halo, C₁₋₄alkyl, C₁₋₄alkoxy, or halo(C₁₋₄)alkyl.

Yet another aspect of the present invention provides a compound of the general formula (1) wherein R⁸ and R⁹ can be H, halo, C₁₋₄alkyl, C₁₋₄alkoxy, halo(C₁₋₄)alkyl, CN, C₁₋₄alkylthio, C₁₋₄alkylsulphinyl, C₁₋₄alkylsulphonyl, aryl, heteroaryl, halo(C₁₋₆)alkoxy, halo(C₁₋₄)alkylthio, C₂₋₄alkenyl, C₂₋₆alkynyl, C₃₋₆cycloalkyl, C₁₋₆alkoxycarbonyl, R³R⁴NCO, NR¹⁰R¹¹, or R⁸ and R⁹ can be joined to form a saturated or unsaturated 5-7 membered carbocyclic or heterocyclic ring, optionally substituted with one or two substituents R¹² where the heterocyclic ring can contain from one to three heteroatoms chosen from NR¹³, O or S;

R¹⁰ and R¹¹, independently of each other, are H or C₁₋₄alkyl;
R¹² is H, halo, C₁₋₄alkyl;
R¹³ is H, C₁₋₄alkyl or halo(C₁₋₄)alkyl

### R² is NR³R⁴;

R is halo chloro or fluoro;
R¹ is phenyl, biphenyl, stilbenzyl or diphenylethynyl optionally substituted with from one to five halogen atoms, particularly fluorine and chlorine atoms and especially fluorine atoms, or with from one to three substituents selected from halo (for example fluoro and chloro), C₁₋₄ alkyl (for example methyl), halo(C₁₋₄)alkyl (for example trifluoromethyl), C₁₋₄ alkoxy (for example methoxy) or halo(C₁₋₄)alkoxy (for example trifluoromethoxy); or R¹ is pyridyl optionally substituted with from one to four halogen atoms or with from one to three substituents selected from halo (for example fluoro and chloro), C₁₋₄ alkyl (for example methyl), halo(C₁₋₄)alkyl (for example trifluoromethyl), C₁₋₄ alkoxy (for example methoxy), halo(C₁₋₄)alkoxy (for example trifluoromethoxy);
R³ and R⁴ are independently H, C₁₋₈ alkyl, C₁₋₈ haloalkyl, C₂₋₈ alkenyl, C₂₋₈ alkynyl, C₃₋₈ cycloalkyl, C₃₋₈ halocycloalkyl, C₃₋₈ cycloalkyl(C₁₋₆)alkyl, or
R³ and R⁴ together form a C₃₋₇ alkylene chain optionally substituted with a C₁₋₄ alkyl group, provided that not both of R⁸ and R⁹ are independently H, halo, C₁₋₄alkyl, C₁₋₄alkoxy, or halo(C₁₋₄)alkyl.

A further aspect of the present invention provides a compound of the general formula (1) wherein R⁸ and R⁹ can be H, halo, C₁₋₄alkyl, C₁₋₄alkoxy, halo(C₁₋₄)alkyl, CN, C₁₋₄alkylthio, aryl, haloalkyl, C₂₋₄alkenyl, C₂₋₆alkynyl, C₃₋₆cycloalkyl, NR¹⁰R¹¹, or R⁸ and R⁹ can be joined to form a saturated or unsaturated 5-7 membered carbocyclic or heterocyclic ring, optionally substituted with one or two substituents R¹², where the heterocyclic ring can contain from one to three heteroatoms chosen from NR¹³, O or S;

R¹⁰ and R¹¹, independently of each other, are H or C₁₋₄alkyl;
R¹² is H, halo, C₁₋₄alkyl,
R¹³ is H or C₁₋₄alkyl;

### R² is NR³R⁴;

R is chloro or fluoro;
R¹ is phenyl or pyridyl, optionally substituted with from one to five chlorine and/or fluorine atoms, or with from one to three substituents selected from halo (for example fluoro and chloro), C₁₋₄ alkyl (for example methyl), halo(C₁₋₄)alkyl (for example trifluoromethyl), C₁₋₄ alkoxy (for example methoxy) or halo(C₁₋₄)alkoxy (for example trifluoromethoxy);
R³ and R⁴ are independently H, C₁₋₈ alkyl, C₁₋₈ haloalkyl, C₃₋₈ cycloalkyl, C₃₋₃ halocycloalkyl, or R³ and R⁴ together form a C₃₋₇ alkylene chain optionally substituted with a C₁₋₄ alkyl group, provided that not both of R⁸ and R⁹ are independently H, halo, C₁₋₄alkyl, C₁₋₄alkoxy, or halo(C₁₋₄)alkyl.

A further embodiment of the present invention provides a compound of the general formula (1) having a structure selected from formulae (1a), (1b), (1c), (1d), (1e), (1f) or (1g), wherein R⁸ can be halo, C₁₋₄alkyl, C₁₋₄alkoxy, halo(C₁₋₄)alkyl, CN, C₁₋₄alkylthio, aryl, haloalkyl, C₂₋₄alkenyl, C₂₋₆alkynyl, C₃₋₆cycloalkyl, NR¹⁰R¹¹, R¹⁰ and R¹¹, independently of each other, are H or C₁₋₄alkyl;

### R² is NR³R⁴;

R is chloro or fluoro;
R¹ is phenyl or pyridyl, optionally substituted with from one to five chlorine and/or fluorine atoms, or with from one to three substituents selected from halo (for example fluoro and chloro), C₁₋₄ alkyl (for example methyl), halo(C₁₋₄)alkyl (for example trifluoromethyl), C₁₋₄ alkoxy (for example methoxy) or halo(C₁₋₄)alkoxy (for example trifluoromethoxy);
R³ and R⁴ are independently H, C₁₋₈ alkyl, C₁₋₈ haloalkyl (for example, C₁₋₈ fluoroalkyl or C₁₋₈ perfluoroalkyl), C₃₋₈ cycloalkyl, C₃₋₈ halocycloalkyl, or R³ and R⁴ together form a C₃₋₇ alkylene chain optionally substituted with a C₁₋₄ alkyl group, provided that not both of R⁸ and R⁹ are independently H, halo, C₁₋₄alkyl, C₁₋₄alkoxy, or halo(C₁₋₄)alkyl.

Another embodiment of the present invention provides a compound of the general formula (1) having a structure selected from formulae (1a), (1b), (1c), (1d), (1e), (1f) or (1g), wherein R⁸ can be chloro, fluoro, methyl, ethyl, n- and iso-propyl, n-, sec-, iso- and tert-butyl, n-pentyl, n-hexyl, CN, methylthio, ethylthio, n- and isopropylthio, phenyl, 4-chlorophenyl, 3-chlorophenyl, 2-chlorophenyl, 4-fluorophenyl, 3-fluorophenyl, 2-fluorophenyl, 2,6-difluorophenyl, 2-fluoro-6-chlorophenyl, 2,5,6-trifluorophenyl, 2,4,6-trifluorophenyl, 2,6-difluoro-4-methoxyphenyl, pentafluorophenyl, 2-fluorophenyl, 2,3,5,6-tetrafluorophenyl, 2-chloro-4,6-difluorophenyl, 2-chlorophenyl, 2,6-dichlorophenyl, 2,4-dichlorophenyl, 2,4,6-trichlorophenyl, 2,3,6-trichlorophenyl, pentachlorophenyl, 2-fluoro-4,6-dichlorophenyl, 4-fluoro-2,6-dichlorophenyl, 2-bromophenyl, 2-fluoro-6-bromophenyl, 2-bromo-4,6-difluorophenyl, vinyl, 1-propen-1-yl, 2-propen-1-yl, ethynyl, propargyl, cyclopropyl, cyclobutyl, cyclopentyl, cyclohexyl, amino, methylamino, dimethylamino, ethylamino, diethylamino;

### R² is NR³R⁴;

R is chloro or fluoro;
R¹ is selected from the group comprising phenyl, 4-chlorophenyl, 3-chlorophenyl, 2-chlorophenyl, 4-fluorophenyl, 3-fluorophenyl, 2-fluorophenyl, 2,6-difluorophenyl, 2-fluoro-6-chlorophenyl, 2,5,6-trifluorophenyl, 2,4,6-trifluorophenyl, 2,6-difluoro-4-methoxyphenyl, pentafluorophenyl, 2-fluorophenyl, 2,3,5,6-tetrafluorophenyl, 2-chloro-4,6-difluorophenyl, 2-chlorophenyl, 2,6-dichlorophenyl, 2,4-dichlorophenyl, 2,4,6-trichlorophenyl, 2,3,6-trichlorophenyl, pentachlorophenyl, 2-fluoro-4,6-dichlorophenyl, 4-fluoro-2,6-dichlorophenyl, 2-bromophenyl, 2-fluoro-6-bromophenyl, 2-bromo-4,6-difluorophenyl, 2-fluoro-6-methylphenyl, 2-chloro-6-methylphenyl, 2-methoxyphenyl, 2,6-dimethoxyphenyl, 2-fluoro-6-methoxyphenyl, 2-trifluoromethylphenyl, 2-fluoro-6-trifluoromethylphenyl, 2,6-di-(trifluoromethyl)phenyl, 2-chloro-6-trifluoromethylphenyl, 2,4-difluoro-6-trifluoromethylphenyl, 2,4-difluoro-6-methoxyphenyl and 2,4-difluoro-6-methylphenyl, 2,4-difluoropyrid-3-yl, 3,5-difluoropyrid-4-yl, tetrafluoropyrid-4-yl, 3-fluoropyrid-2-yl, 4-fluoropyrid-3-yl, 3-fluoropyrid-4-yl, 2-fluoropyrid-3-yl, 2,4,6-trifluoropyrid-3-yl, 3,5-difluoropyrid-2-yl, 2,6-difluoropyrid-3-yl, 2,4-difluoro-6-methoxypyrid-3-yl, 2-fluoro-4-chloropyrid-3-yl, 3-fluoro-5-chloropyrid-4-yl, 2-chloro-4-fluoropyrid-3-yl, 2,4-dichloropyrid-3-yl, 3-chloropyrid-2-yl 1,4-chloropyrid-3-yl, 3-chloropyrid-4-yl, 2-chloropyrid-3-yl, 3-trifluoromethylpyrid-2-yl, 4-trifluoromethylpyrid-3-yl, 3,5-dichloropyrid-2-yl, 3-fluoro-5-chloropyrid-2-yl, 4,6-dichloropyrid-3-yl, 3-trifluoromethylpyrid-4-yl, 2-trifluoromethylpyrid-3-yl, 2-fluoro-4-trifluoromethylpyrid-3-yl, 3-fluoro-5-trifluoromethylpyrid-4-yl, 4-fluoro-2-trifluoromethylpyrid-3-yl, 2,6-dichloropyrid-3-yl, 3,5-dichloropyrid-4-yl, 3-chloro-5-trifluoromethylpyrid-2-yl, 3-fluoro-6-trifluoromethylpyrid-2-yl, pyrid-2-yl, pyrid-3-yl and pyrid-4-yl;
R³ is hydrogen, methyl, ethyl, 1,1,1-trifluoroethyl, 2-methylpropen-3-yl;
R⁴ is prop-2-yl, but-2-yl, 2-methylprop-3-yl, 2-methylbut-3-yl, 1,1,1-trifluoroprop-2-yl, 1,1,1-trifluoroethyl, 1,1,1-trifluorobut-2-yl, 1,1,1-trifluoro-3-methyl-but-2-yl; 2-methylpent-4-yl, 1,1,1-trifluoro-4-methylpent-2-yl, 1,1,1-trifluoro-3-methylpent-2-yl, 3-methylpent-2-yl, 1,1,-difluorocyclopent-2-yl, heptafluoroprop-1-yl, nonafluorobut-1-yl, 1-carboxyethyl-2.methylprop-1-yl, 1-carboxymethyl-2-methylprop-1-yl, 1,1,1,-trifluoro-2-carboxyethyl-2-ethyl, 1,1,1,-trifluoro-2-carboxymethyl 2-ethyl;
or together with the nitrogen atom to which they are attached, R³ and R⁴ form a morpholine ring or a piperidine, piperazine or piperazine N—(C₁₋₄)alkyl (especially N-methyl) ring.

In Table 1 the compounds have the general formula (1h), where R, R¹, R³ and R⁴ are as shown in the table, R⁸ is H, R⁹ is —CN. Characterising data is given later in the Examples. Single compounds are assigned the number of the table, followed by the number of the combination of substituents as in Table 1. For example, compound 22.005 is the compound as described in Table 22, wherein the substituents defined therein are combined with the substituents as defined in Table 1, position No. 5.

TABLE 1

| | |
|---|---|
| | (1h) |
| 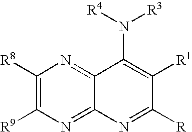 | |

| Compound | | | | |
|---|---|---|---|---|
| No. | R | R¹ | R³ | R⁴ |
| 1.001 | Cl | 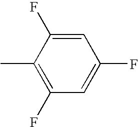 | H | —CH(CH₃)CH₂CH₃ |
| 1.002 | Cl | 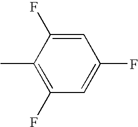 | H | —CH₂CH(CH₃)₂ |
| 1.003 | Cl | 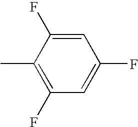 | H | —CH(CH₃)CH(CH₃)₂ |
| 1.004 | Cl | 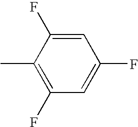 | H | —CH(CH₃)(CF₃) |
| 1.005 | Cl | 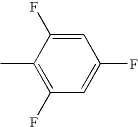 | H | —CH(CF₃)CH₂CH₃ |
| 1.006 | Cl | 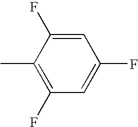 | H | (R) —CH(CF₃)CH₂CH₃ |
| 1.007 | Cl | 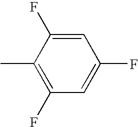 | H | (S) —CH(CF₃)CH₂CH₃ |
| 1.008 | Cl | 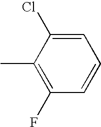 | H | —CH(CH₃)CH₂CH₃ |
| 1.009 | Cl | 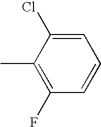 | H | —CH₂CH(CH₃)₂ |
| 1.010 | Cl | 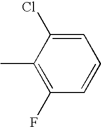 | H | —CH(CH₃)CH(CH₃)₂ |
| 1.011 | Cl | 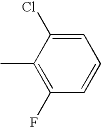 | H | —CH(CH₃)(CF₃) |
| 1.012 | Cl | 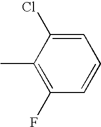 | H | —CH(CF₃)CH₂CH₃ |
| 1.013 | Cl | 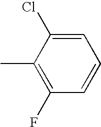 | H | (R) —CH(CF₃)CH₂CH₃ |
| 1.014 | Cl | 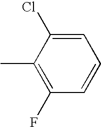 | H | (S) —CH(CF₃)CH₂CH₃ |
| 1.015 | Cl | 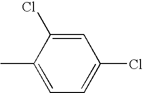 | H | —CH(CH₃)CH₂CH₃ |
| 1.016 | Cl | 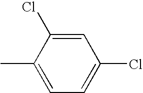 | H | —CH₂CH(CH₃)₂ |
| 1.017 | Cl | 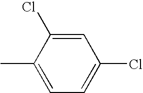 | H | —CH(CH₃)CH(CH₃)₂ |
| 1.018 | Cl | 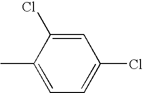 | H | —CH(CH₃)(CF₃) |
| 1.019 | Cl | 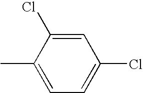 | H | —CH(CF₃)CH₂CH₃ |
| 1.020 | Cl | 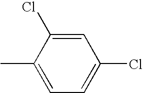 | H | (R) —CH(CF₃)CH₂CH₃ |
| 1.021 | Cl | 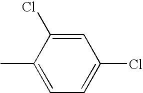 | H | (S) —CH(CF₃)CH₂CH₃ |
| 1.022 | Cl | 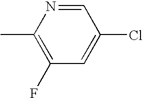 | H | —CH(CH₃)CH₂CH₃ |
| 1.023 | Cl | 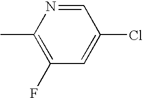 | H | —CH₂CH(CH₃)₂ |
| 1.024 | Cl | 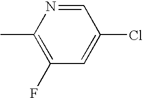 | H | —CH(CH₃)CH(CH₃)₂ |
| 1.025 | Cl | 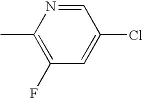 | H | —CH(CH₃)(CF₃) |
| 1.026 | Cl | 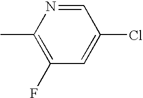 | H | —CH(CF₃)CH₂CH₃ |
| 1.027 | Cl | 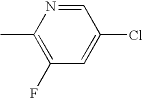 | H | (R) —CH(CF₃)CH₂CH₃ |
| 1.028 | Cl | 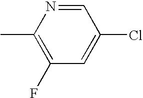 | H | (S) —CH(CF₃)CH₂CH₃ |
| 1.029 | Cl | 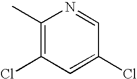 | H | —CH(CH₃)CH₂CH₃ |
| 1.030 | Cl | 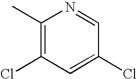 | H | —CH₂CH(CH₃)₂ |
| 1.031 | Cl | 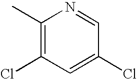 | H | —CH(CH₃)CH(CH₃)₂ |
| 1.032 | Cl | 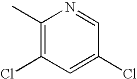 | H | —CH(CH₃)(CF₃) |
| 1.033 | Cl | 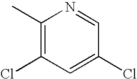 | H | —CH(CF₃)CH₂CH₃ |
| 1.034 | Cl | 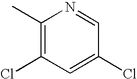 | H | (R) —CH(CF₃)CH₂CH₃ |
| 1.035 | Cl | 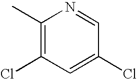 | H | (S) —CH(CF₃)CH₂CH₃ |
| 1.036 | F | 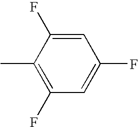 | H | —CH(CH₃)CH₂CH₃ |
| 1.037 | F | 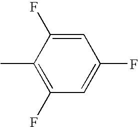 | H | —CH₂CH(CH₃)₂ |
| 1.038 | F | 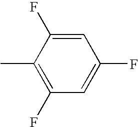 | H | —CH(CH₃)CH(CH₃)₂ |
| 1.039 | F | 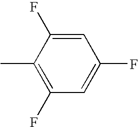 | H | —CH(CH₃)(CF₃) |
| 1.040 | F | 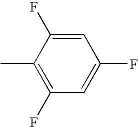 | H | —CH(CF₃)CH₂CH₃ |
| 1.041 | F | 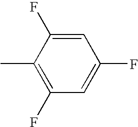 | H | (R) —CH(CF₃)CH₂CH₃ |
| 1.042 | F | 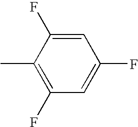 | H | (S) —CH(CF₃)CH₂CH₃ |
| 1.043 | F | 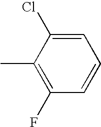 | H | —CH(CH₃)CH₂CH₃ |
| 1.044 | F | 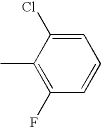 | H | —CH₂CH(CH₃)₂ |
| 1.045 | F | 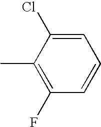 | H | —CH(CH₃)CH(CH₃)₂ |
| 1.046 | F | 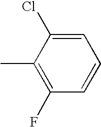 | H | —CH(CH₃)(CF₃) |
| 1.047 | F | 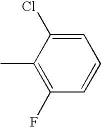 | H | —CH(CF₃)CH₂CH₃ |
| 1.048 | F | 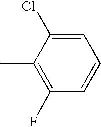 | H | (R) —CH(CF₃)CH₂CH₃ |
| 1.049 | F | 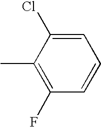 | H | (S) —CH(CF₃)CH₂CH₃ |
| 1.050 | F | 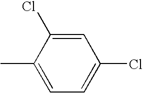 | H | —CH(CH₃)CH₂CH₃ |
| 1.051 | F | 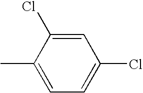 | H | —CH₂CH(CH₃)₂ |
| 1.052 | F | 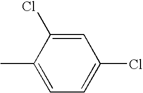 | H | —CH(CH₃)CH(CH₃)₂ |
| 1.053 | F | 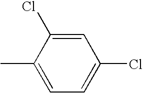 | H | —CH(CH₃)(CF₃) |
| 1.054 | F | 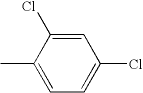 | H | —CH(CF₃)CH₂CH₃ |
| 1.055 | F | 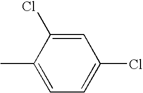 | H | (R) —CH(CF₃)CH₂CH₃ |
| 1.056 | F | 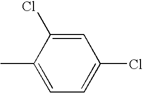 | H | (S) —CH(CF₃)CH₂CH₃ |
| 1.057 | F | 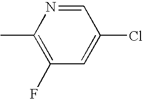 | H | —CH(CH₃)CH₂CH₃ |
| 1.058 | F | 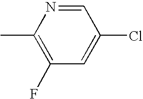 | H | —CH₂CH(CH₃)₂ |
| 1.059 | F | 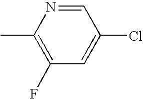 | H | —CH(CH₃)CH(CH₃)₂ |
| 1.060 | F | 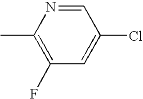 | H | —CH(CH₃)(CF₃) |
| 1.061 | F | 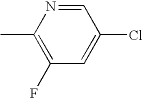 | H | —CH(CF₃)CH₂CH₃ |
| 1.062 | F | 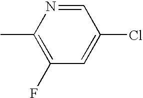 | H | (R) —CH(CF₃)CH₂CH₃ |
| 1.063 | F | 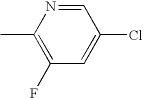 | H | (S) —CH(CF₃)CH₂CH₃ |
| 1.064 | F | 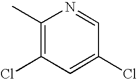 | H | —CH(CH₃)CH₂CH₃ |
| 1.065 | F | 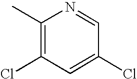 | H | —CH₂CH(CH₃)₂ |
| 1.066 | F | 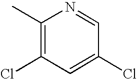 | H | —CH(CH₃)CH(CH₃)₂ |
| 1.067 | F | 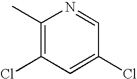 | H | —CH(CH₃)(CF₃) |
| 1.068 | F | 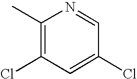 | H | —CH(CF₃)CH₂CH₃ |
| 1.069 | F | 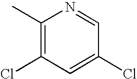 | H | (R) —CH(CF₃)CH₂CH₃ |
| 1.070 | F | 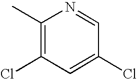 | H | (S) —CH(CF₃)CH₂CH₃ |
| 1.071 | Cl | 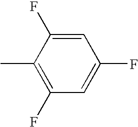 | —CH₂CH₃ | —CH(CH₃)CH₂CH₃ |
| 1.072 | Cl | 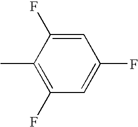 | —CH₂CH₃ | —CH₂CH(CH₃)₂ |
| 1.073 | Cl | 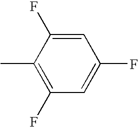 | —CH₂CH₃ | —CH(CH₃)CH(CH₃)₂ |
| 1.074 | Cl | 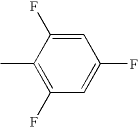 | —CH₂CH₃ | —CH(CH₃)(CF₃) |
| 1.075 | Cl | 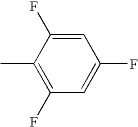 | —CH₂CH₃ | —CH(CF₃)CH₂CH₃ |
| 1.076 | Cl | 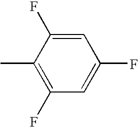 | —CH₂CH₃ | (R) —CH(CF₃)CH₂CH₃ |
| 1.077 | Cl | 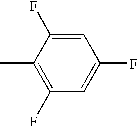 | —CH₂CH₃ | (S) —CH(CF₃)CH₂CH₃ |
| 1.078 | Cl | 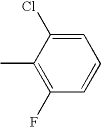 | —CH₂CH₃ | —CH(CH₃)CH₂CH₃ |
| 1.079 | Cl | 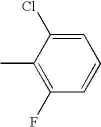 | —CH₂CH₃ | —CH₂CH(CH₃)₂ |
| 1.080 | Cl | 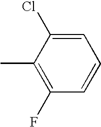 | —CH₂CH₃ | —CH(CH₃)CH(CH₃)₂ |
| 1.081 | Cl | 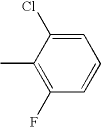 | —CH₂CH₃ | —CH(CH₃)(CF₃) |
| 1.082 | Cl | 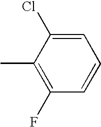 | —CH₂CH₃ | —CH(CF₃)CH₂CH₃ |
| 1.083 | Cl | 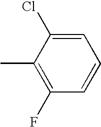 | —CH₂CH₃ | (R) —CH(CF₃)CH₂CH₃ |
| 1.084 | Cl | 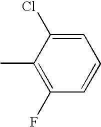 | —CH₂CH₃ | (S) —CH(CF₃)CH₂CH₃ |
| 1.085 | Cl | 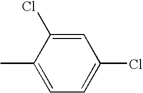 | —CH₂CH₃ | —CH(CH₃)CH₂CH₃ |
| 1.086 | Cl | 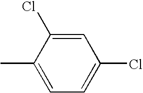 | —CH₂CH₃ | —CH₂CH(CH₃)₂ |
| 1.087 | Cl | 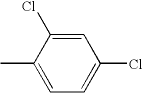 | —CH₂CH₃ | —CH(CH₃)CH(CH₃)₂ |
| 1.088 | Cl | 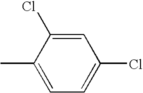 | —CH₂CH₃ | —CH(CH₃)(CF₃) |
| 1.089 | Cl | 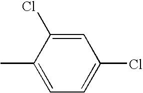 | —CH₂CH₃ | —CH(CF₃)CH₂CH₃ |
| 1.090 | Cl | 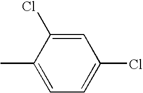 | —CH₂CH₃ | (R) —CH(CF₃)CH₂CH₃ |
| 1.091 | Cl | 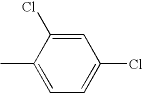 | —CH₂CH₃ | (S) —CH(CF₃)CH₂CH₃ |
| 1.092 | Cl | 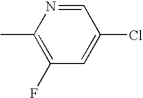 | —CH₂CH₃ | —CH(CH₃)CH₂CH₃ |
| 1.093 | Cl | 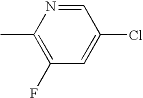 | —CH₂CH₃ | —CH₂CH(CH₃)₂ |
| 1.094 | Cl | 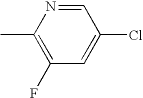 | —CH₂CH₃ | —CH(CH₃)CH(CH₃)₂ |
| 1.095 | Cl | 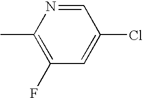 | —CH₂CH₃ | —CH(CH₃)(CF₃) |
| 1.096 | Cl | 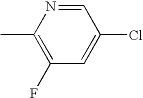 | —CH₂CH₃ | —CH(CF₃)CH₂CH₃ |
| 1.097 | Cl | 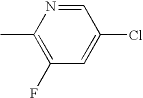 | —CH₂CH₃ | (R) —CH(CF₃)CH₂CH₃ |
| 1.098 | Cl | 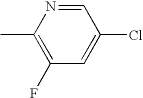 | —CH₂CH₃ | (S) —CH(CF₃)CH₂CH₃ |
| 1.099 | Cl | 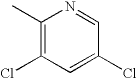 | —CH₂CH₃ | —CH(CH₃)CH₂CH₃ |
| 1.100 | Cl | 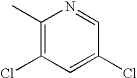 | —CH₂CH₃ | —CH₂CH(CH₃)₂ |
| 1.101 | Cl | 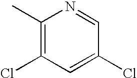 | —CH₂CH₃ | —CH(CH₃)CH(CH₃)₂ |
| 1.102 | Cl | 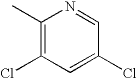 | —CH₂CH₃ | —CH(CH₃)(CF₃) |
| 1.103 | Cl | 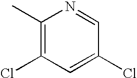 | —CH₂CH₃ | —CH(CF₃)CH₂CH₃ |
| 1.104 | Cl | 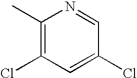 | —CH₂CH₃ | (R) —CH(CF₃)CH₂CH₃ |
| 1.105 | Cl | 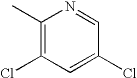 | —CH₂CH₃ | (S) —CH(CF₃)CH₂CH₃ |
| 1.106 | F | 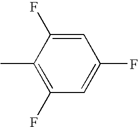 | —CH₂CH₃ | —CH(CH₃)CH₂CH₃ |
| 1.107 | F | 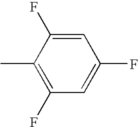 | —CH₂CH₃ | —CH₂CH(CH₃)₂ |
| 1.108 | F | 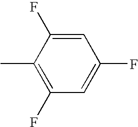 | —CH₂CH₃ | —CH(CH₃)CH(CH₃)₂ |
| 1.109 | F | 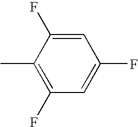 | —CH₂CH₃ | —CH(CH₃)(CF₃) |
| 1.110 | F | 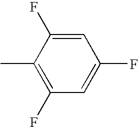 | —CH₂CH₃ | —CH(CF₃)CH₂CH₃ |
| 1.111 | F | 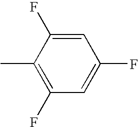 | —CH₂CH₃ | (R) —CH(CF₃)CH₂CH₃ |
| 1.112 | F | 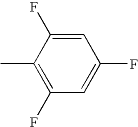 | —CH₂CH₃ | (S) —CH(CF₃)CH₂CH₃ |
| 1.113 | F | 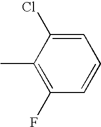 | —CH₂CH₃ | —CH(CH₃)CH₂CH₃ |
| 1.114 | F | 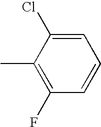 | —CH₂CH₃ | —CH₂CH(CH₃)₂ |
| 1.115 | F | 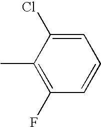 | —CH₂CH₃ | —CH(CH₃)CH(CH₃)₂ |
| 1.116 | F | 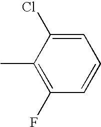 | —CH₂CH₃ | —CH(CH₃)(CF₃) |
| 1.117 | F | 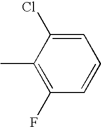 | —CH₂CH₃ | —CH(CF₃)CH₂CH₃ |
| 1.118 | F | 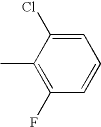 | —CH₂CH₃ | (R) —CH(CF₃)CH₂CH₃ |
| 1.119 | F | 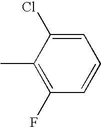 | —CH₂CH₃ | (S) —CH(CF₃)CH₂CH₃ |
| 1.120 | F | 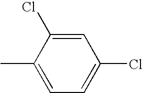 | —CH₂CH₃ | —CH(CH₃)CH₂CH₃ |
| 1.121 | F | 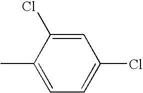 | —CH₂CH₃ | —CH₂CH(CH₃)₂ |
| 1.122 | F | 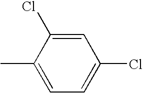 | —CH₂CH₃ | —CH(CH₃)CH(CH₃)₂ |
| 1.123 | F | 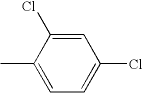 | —CH₂CH₃ | —CH(CH₃)(CF₃) |
| 1.124 | F | 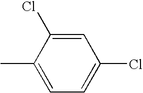 | —CH₂CH₃ | —CH(CF₃)CH₂CH₃ |
| 1.125 | F | 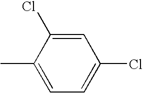 | —CH₂CH₃ | (R) —CH(CF₃)CH₂CH₃ |
| 1.126 | F | 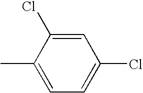 | —CH₂CH₃ | (S) —CH(CF₃)CH₂CH₃ |
| 1.127 | F | 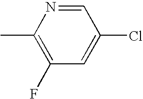 | —CH₂CH₃ | —CH(CH₃)CH₂CH₃ |
| 1.128 | F | 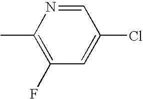 | —CH₂CH₃ | —CH₂CH(CH₃)₂ |
| 1.129 | F | 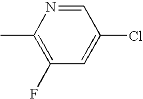 | —CH₂CH₃ | —CH(CH₃)CH(CH₃)₂ |
| 1.130 | F | 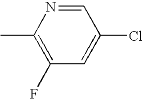 | —CH₂CH₃ | —CH(CH₃)(CF₃) |
| 1.131 | F | 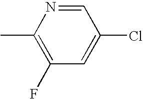 | —CH₂CH₃ | —CH(CF₃)CH₂CH₃ |
| 1.132 | F | 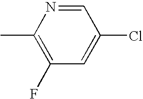 | —CH₂CH₃ | (R) —CH(CF₃)CH₂CH₃ |
| 1.133 | F | 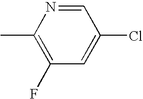 | —CH₂CH₃ | (S) —CH(CF₃)CH₂CH₃ |
| 1.134 | F | 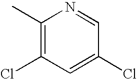 | —CH₂CH₃ | —CH(CH₃)CH₂CH₃ |
| 1.135 | F | 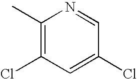 | —CH₂CH₃ | —CH₂CH(CH₃)₂ |
| 1.136 | F | 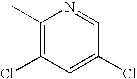 | —CH₂CH₃ | —CH(CH₃)CH(CH₃)₂ |
| 1.137 | F | 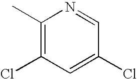 | —CH₂CH₃ | —CH(CH₃)(CF₃) |
| 1.138 | F | 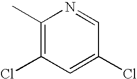 | —CH₂CH₃ | —CH(CF₃)CH₂CH₃ |
| 1.139 | F | 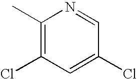 | —CH₂CH₃ | (R) —CH(CF₃)CH₂CH₃ |
| 1.140 | F | 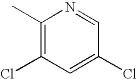 | —CH₂CH₃ | (S) —CH(CF₃)CH₂CH₃ |
| 1.141 | Cl | 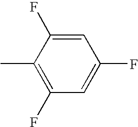 | —CH₂C(CH₃)═CH₂ | —CH(CH₃)CH₂CH₃ |
| 1.142 | Cl | 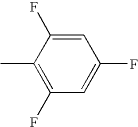 | —CH₂C(CH₃)═CH₂ | —CH₂CH(CH₃)₂ |
| 1.143 | Cl | 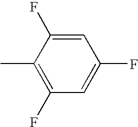 | —CH₂C(CH₃)═CH₂ | —CH(CH₃)CH(CH₃)₂ |
| 1.144 | Cl | 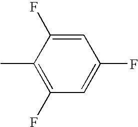 | —CH₂C(CH₃)═CH₂ | —CH(CH₃)(CF₃) |
| 1.145 | Cl | 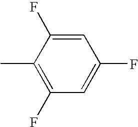 | —CH₂C(CH₃)═CH₂ | —CH(CF₃)CH₂CH₃ |
| 1.146 | Cl | 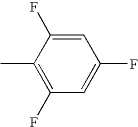 | —CH₂C(CH₃)═CH₂ | (R) —CH(CF₃)CH₂CH₃ |
| 1.147 | Cl | 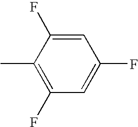 | —CH₂C(CH₃)═CH₂ | (S) —CH(CF₃)CH₂CH₃ |
| 1.148 | Cl | 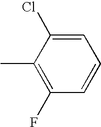 | —CH₂C(CH₃)═CH₂ | —CH(CH₃)CH₂CH₃ |
| 1.149 | Cl | 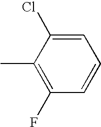 | —CH₂C(CH₃)═CH₂ | —CH₂CH(CH₃)₂ |
| 1.150 | Cl | 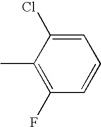 | —CH₂C(CH₃)═CH₂ | —CH(CH₃)CH(CH₃)₂ |
| 1.151 | Cl | 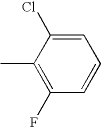 | —CH₂C(CH₃)═CH₂ | —CH(CH₃)(CF₃) |
| 1.152 | Cl | 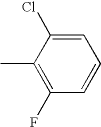 | —CH₂C(CH₃)═CH₂ | —CH(CF₃)CH₂CH₃ |
| 1.153 | Cl | 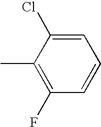 | —CH₂C(CH₃)═CH₂ | (R) —CH(CF₃)CH₂CH₃ |
| 1.154 | Cl | 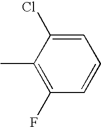 | —CH₂C(CH₃)═CH₂ | (S) —CH(CF₃)CH₂CH₃ |
| 1.155 | Cl | 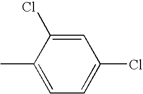 | —CH₂C(CH₃)═CH₂ | —CH(CH₃)CH₂CH₃ |
| 1.156 | Cl | 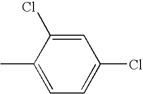 | —CH₂C(CH₃)═CH₂ | —CH₂CH(CH₃)₂ |
| 1.157 | Cl | 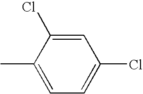 | —CH₂C(CH₃)═CH₂ | —CH(CH₃)CH(CH₃)₂ |
| 1.158 | Cl | 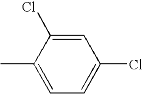 | —CH₂C(CH₃)═CH₂ | —CH(CH₃)(CF₃) |
| 1.159 | Cl | 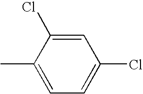 | —CH₂C(CH₃)═CH₂ | —CH(CF₃)CH₂CH₃ |
| 1.160 | Cl | 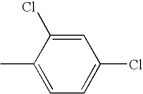 | —CH₂C(CH₃)═CH₂ | (R) —CH(CF₃)CH₂CH₃ |
| 1.161 | Cl | 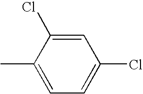 | —CH₂C(CH₃)═CH₂ | (S) —CH(CF₃)CH₂CH₃ |
| 1.162 | Cl | 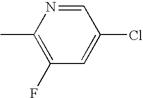 | —CH₂C(CH₃)═CH₂ | —CH(CH₃)CH₂CH₃ |
| 1.163 | Cl | 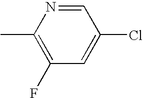 | —CH₂C(CH₃)═CH₂ | —CH₂CH(CH₃)₂ |
| 1.164 | Cl | 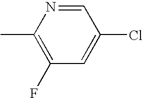 | —CH₂C(CH₃)═CH₂ | —CH(CH₃)CH(CH₃)₂ |
| 1.165 | Cl | 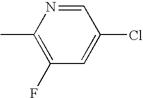 | —CH₂C(CH₃)═CH₂ | —CH(CH₃)(CF₃) |
| 1.166 | Cl | 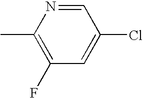 | —CH₂C(CH₃)═CH₂ | —CH(CF₃)CH₂CH₃ |
| 1.167 | Cl | 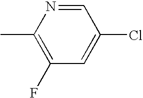 | —CH₂C(CH₃)═CH₂ | (R) —CH(CF₃)CH₂CH₃ |
| 1.168 | Cl | 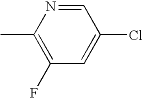 | —CH₂C(CH₃)═CH₂ | (S) —CH(CF₃)CH₂CH₃ |
| 1.169 | Cl | 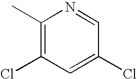 | —CH₂C(CH₃)═CH₂ | —CH(CH₃)CH₂CH₃ |
| 1.170 | Cl | 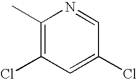 | —CH₂C(CH₃)═CH₂ | —CH₂CH(CH₃)₂ |
| 1.171 | Cl | 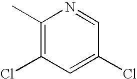 | —CH₂C(CH₃)═CH₂ | —CH(CH₃)CH(CH₃)₂ |
| 1.172 | Cl | 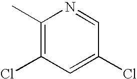 | —CH₂C(CH₃)═CH₂ | —CH(CH₃)(CF₃) |
| 1.173 | Cl | 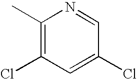 | —CH₂C(CH₃)═CH₂ | —CH(CF₃)CH₂CH₃ |
| 1.174 | Cl | 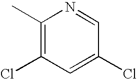 | —CH₂C(CH₃)═CH₂ | (R) —CH(CF₃)CH₂CH₃ |
| 1.175 | Cl | 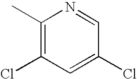 | —CH₂C(CH₃)═CH₂ | (S) —CH(CF₃)CH₂CH₃ |
| 1.176 | F | 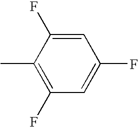 | —CH₂C(CH₃)═CH₂ | —CH(CH₃)CH₂CH₃ |
| 1.177 | F | 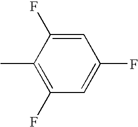 | —CH₂C(CH₃)═CH₂ | —CH₂CH(CH₃)₂ |
| 1.178 | F | 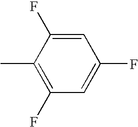 | —CH₂C(CH₃)═CH₂ | —CH(CH₃)CH(CH₃)₂ |
| 1.179 | F | 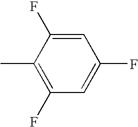 | —CH₂C(CH₃)═CH₂ | —CH(CH₃)(CF₃) |
| 1.180 | F | 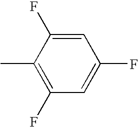 | —CH₂C(CH₃)═CH₂ | —CH(CF₃)CH₂CH₃ |
| 1.181 | F | 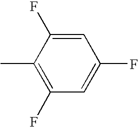 | —CH₂C(CH₃)═CH₂ | (R) —CH(CF₃)CH₂CH₃ |
| 1.182 | F | 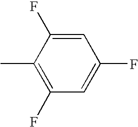 | —CH₂C(CH₃)═CH₂ | (S) —CH(CF₃)CH₂CH₃ |
| 1.183 | F | 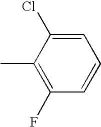 | —CH₂C(CH₃)═CH₂ | —CH(CH₃)CH₂CH₃ |
| 1.184 | F | 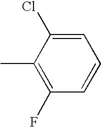 | —CH₂C(CH₃)═CH₂ | —CH₂CH(CH₃)₂ |
| 1.185 | F | 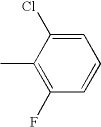 | —CH₂C(CH₃)═CH₂ | —CH(CH₃)CH(CH₃)₂ |
| 1.186 | F | 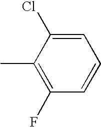 | —CH₂C(CH₃)═CH₂ | —CH(CH₃)(CF₃) |
| 1.187 | F | 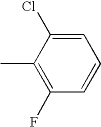 | —CH₂C(CH₃)═CH₂ | —CH(CF₃)CH₂CH₃ |
| 1.188 | F | 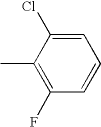 | —CH₂C(CH₃)═CH₂ | (R) —CH(CF₃)CH₂CH₃ |
| 1.189 | F | 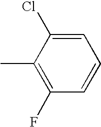 | —CH₂C(CH₃)═CH₂ | (S) —CH(CF₃)CH₂CH₃ |
| 1.190 | F | 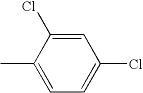 | —CH₂C(CH₃)═CH₂ | —CH(CH₃)CH₂CH₃ |
| 1.191 | F | 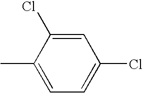 | —CH₂C(CH₃)═CH₂ | —CH₂CH(CH₃)₂ |
| 1.192 | F | 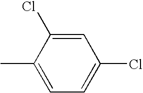 | —CH₂C(CH₃)═CH₂ | —CH(CH₃)CH(CH₃)₂ |
| 1.193 | F | 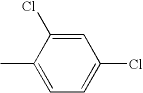 | —CH₂C(CH₃)═CH₂ | —CH(CH₃)(CF₃) |
| 1.194 | F | 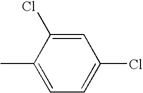 | —CH₂C(CH₃)═CH₂ | —CH(CF₃)CH₂CH₃ |
| 1.195 | F | 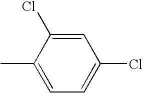 | —CH₂C(CH₃)═CH₂ | (R) —CH(CF₃)CH₂CH₃ |
| 1.196 | F | 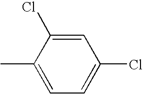 | —CH₂C(CH₃)═CH₂ | (S) —CH(CF₃)CH₂CH₃ |
| 1.197 | F | 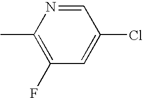 | —CH₂C(CH₃)═CH₂ | —CH(CH₃)CH₂CH₃ |
| 1.198 | F | 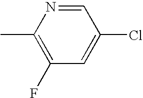 | —CH₂C(CH₃)═CH₂ | —CH₂CH(CH₃)₂ |
| 1.199 | F | 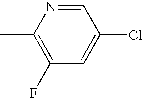 | —CH₂C(CH₃)═CH₂ | —CH(CH₃)CH(CH₃)₂ |
| 1.200 | F | 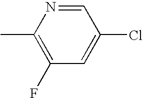 | —CH₂C(CH₃)═CH₂ | —CH(CH₃)(CF₃) |
| 1.201 | F | 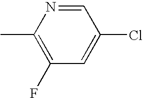 | —CH₂C(CH₃)═CH₂ | —CH(CF₃)CH₂CH₃ |
| 1.202 | F | 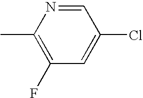 | —CH₂C(CH₃)═CH₂ | (R) —CH(CF₃)CH₂CH₃ |
| 1.203 | F | 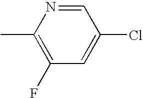 | —CH₂C(CH₃)═CH₂ | (S) —CH(CF₃)CH₂CH₃ |
| 1.204 | F | 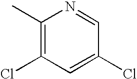 | —CH₂C(CH₃)═CH₂ | —CH(CH₃)CH₂CH₃ |
| 1.205 | F | 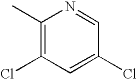 | —CH₂C(CH₃)═CH₂ | —CH₂CH(CH₃)₂ |
| 1.206 | F | 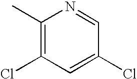 | —CH₂C(CH₃)═CH₂ | —CH(CH₃)CH(CH₃)₂ |
| 1.207 | F | 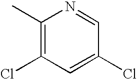 | —CH₂C(CH₃)═CH₂ | —CH(CH₃)(CF₃) |
| 1.208 | F | 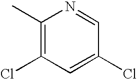 | —CH₂C(CH₃)═CH₂ | —CH(CF₃)CH₂CH₃ |
| 1.209 | F | 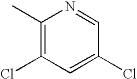 | —CH₂C(CH₃)═CH₂ | (R) —CH(CF₃)CH₂CH₃ |
| 1.210 | F | 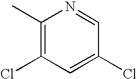 | —CH₂C(CH₃)═CH₂ | (S) —CH(CF₃)CH₂CH₃ |

### Table 2

Table 2 consists of 210 compounds of the general formula (1h), where R, R¹, R³ and R⁴ are as listed in Table 1, R⁸ is H, R⁹ is —CN.

### Table 3

Table 3 consists of 210 compounds of the general formula (1h), where R, R¹, R³ and R⁴ are as listed in Table 1, R³ is H, R⁹ is CH₂═CH—.

### Table 4

Table 4 consists of 210 compounds of the general formula (1h), where R, R¹, R³ and R⁴ are as listed in Table 1, R⁸ is H, R⁹ is CH₃S—.

### Table 5

Table 5 consists of 210 compounds of the general formula (1h), where R, R¹, R³ and R⁴ are as listed in Table 1, R⁸ is H, R⁹ is —NH₂.

### Table 6

Table 6 consists of 210 compounds of the general formula (1h), where R, R¹, R³ and R⁴ are as listed in Table 1, R⁸ is H, R⁹ is (CH₃)₂N—.

### Table 7

Table 7 consists of 210 compounds of the general formula (1h), where R, R¹, R³ and R⁴ are as listed in Table 1, R⁸ is H, R⁹ is ethynyl.

### Table 8

Table 8 consists of 210 compounds of the general formula (1h), where R, R¹, R³ and R⁴ are as listed in Table 1, R⁸ is H, R⁹ is cyclopropyl.

### Table 9

Table 9 consists of 210 compounds of the general formula (1h), where R, R¹, R³ and R⁴ are as listed in Table 1, R⁸ is H, R⁹ is 4-fluorophenyl.

### Table 10

Table 10 consists of 210 compounds of the general formula (1h), where R, R¹, R³ and R⁴ are as listed in Table 1, R⁸ is H, R⁹ is phenyl.

### Table 11

Table 11 consists of 210 compounds of the general formula (1h), where R, R¹, R³ and R⁴ are as listed in Table 1, R⁸ is —CN, R⁹ is H.

### Table 12

Table 12 consists of 210 compounds of the general formula (1h), where R, R¹, R³ and R⁴ are as listed in Table 1, R⁸ is CH₂═CH—, R⁹ is H.

### Table 13

Table 13 consists of 210 compounds of the general formula (1h), where R, R¹, R³ and R⁴ are as listed in Table 1, R⁸ is CH₃S—, R⁹ is H.

### Table 14

Table 14 consists of 210 compounds of the general formula (1h), where R, R¹, R³ and R⁴ are as listed in Table 1, R⁸ is —NH₂, R⁹ is H.

### Table 15

Table 15 consists of 210 compounds of the general formula (1h), where R, R¹, R³ and R⁴ are as listed in Table 1, R⁸ is (CH₃)₂N—, R⁹ is H.

### Table 16

Table 16 consists of 210 compounds of the general formula (1h), where R, R¹, R³ and R⁴ are as listed in Table 1, R⁸ is ethynyl, R⁹ is H.

### Table 17

Table 17 consists of 210 compounds of the general formula (1h), where R, R¹, R³ and R⁴ are as listed in Table 1, R⁸ is cyclopropyl, R⁹ is H.

### Table 18

Table 18 consists of 210 compounds of the general formula (1h), where R, R¹, R³ and R⁴ are as listed in Table 1, R⁸ is 4-fluorophenyl, R⁹ is H.

### Table 19

Table 19 consists of 210 compounds of the general formula (1h), where R, R¹, R³ and R⁴ are as listed in Table 1, R⁸ is phenyl, R⁹ is H.

### Table 20

Table 20 consists of 210 compounds of the general formula (1h), where R, R¹, R² is NR³R⁴, R³ and R⁴ are as listed in Table 1, and the general formula (1h) is identical to general formula (1a), that is, R⁸ and R⁹ form a methyloxazole ring as depicted below.

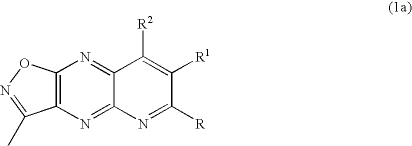

### Table 21

Table 21 consists of 210 compounds of the general formula (1h), where R, R¹, R² is NR³R⁴, R³ and R⁴ are as listed in Table 1, and the general formula (1h) is identical to general formula (1b), that is, R⁸ and R⁹ form a cyclopentyl ring as depicted below.

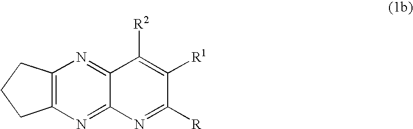

### Table 22

Table 22 consists of 210 compounds of the general formula (1h), where R, R¹, R² is NR³R⁴, R³ and R⁴ are as listed in Table 1, and the general formula (1h) is identical to general formula (1c), that is, R⁸ and R⁹ form a cyclohexyl ring oxazole ring as depicted below.

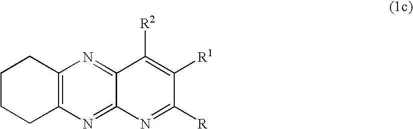

### Table 23

Table 23 consists of 210 compounds of the general formula (1h), where R, R¹, R² is NR³R⁴, R³ and R⁴ are as listed in Table 1, and the general formula (1h) is identical to general formula (1d), that is, R⁸ and R⁹ form a thiazole ring as depicted below.

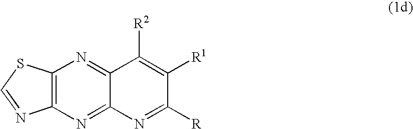

### Table 24

Table 24 consists of 210 compounds of the general formula (1h), where R, R¹, R² is NR³R⁴, R³ and R⁴ are as listed in Table 1, and the general formula (1h) is identical to general formula (1e), that is, R⁸ and R⁹ form an thiazole ring as depicted below.

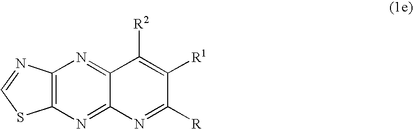

### Table 25

Table 25 consists of 210 compounds of the general formula (1h), where R, R¹, R² is NR³R⁴, R³ and R⁴ are as listed in Table 1, and the general formula (1h) is identical to general formula (1f), that is, R³ and R⁹ form a methylpyrazole ring as depicted below.

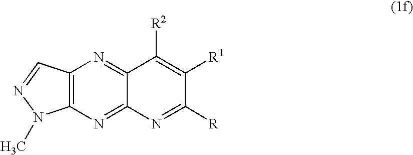

In Table 26 the compounds have the general formula (1k), where R, R¹, R⁸ and R⁹ are as shown in the table. Single compounds are assigned the number of the table, followed by the number of the combination of substituents as in Table 26. For example, compound 27.005 is the compound as described in Table 26, wherein the substituents defined therein are combined with the substituents as defined in Table 1, position No. 5.

TABLE 26

| | |
|---|---|
| | (1k) |
| 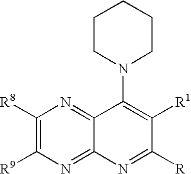 | |

| Compound No. | R | R¹ | R⁸ | R⁹ |
|---|---|---|---|---|
| 26.001 | Cl | 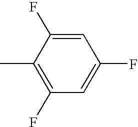 | H | —CN |
| 26.002 | Cl | 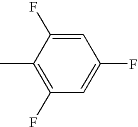 | H | —CH═CH₂ |
| 26.003 | Cl | 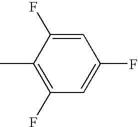 | H | —SCH₃ |
| 26.004 | Cl | 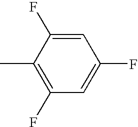 | H | —NH₂ |
| 26.005 | Cl | 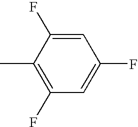 | H | —N(CH₃)₂ |
| 26.006 | Cl | 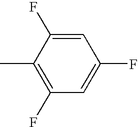 | H | ethynyl |
| 26.007 | Cl | 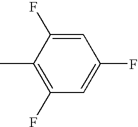 | H | cyclopropyl |
| 26.008 | Cl | 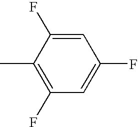 | H | 4-fluorophenyl |
| 26.009 | Cl | 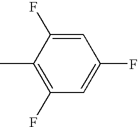 | H | phenyl |
| 26.010 | Cl | 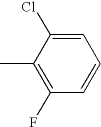 | H | —CN |
| 26.011 | Cl | 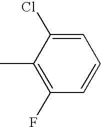 | H | —CH═CH₂ |
| 26.012 | Cl | 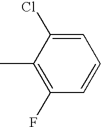 | H | —SCH₃ |
| 26.013 | Cl | 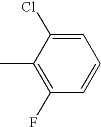 | H | —NH₂ |
| 26.014 | Cl | 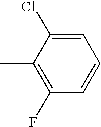 | H | —N(CH₃)₂ |
| 26.015 | Cl | 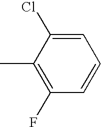 | H | ethynyl |
| 26.016 | Cl | 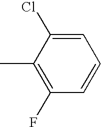 | H | cyclopropyl |
| 26.017 | Cl | 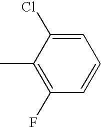 | H | 4-fluorophenyl |
| 26.018 | Cl | 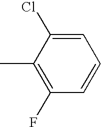 | H | phenyl |
| 26.019 | Cl | 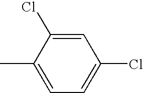 | H | —CN |
| 26.020 | Cl | 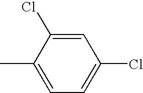 | H | —CH═CH₂ |
| 26.021 | Cl | 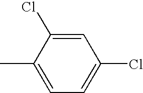 | H | —SCH₃ |
| 26.022 | Cl | 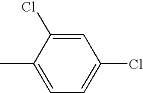 | H | —NH₂ |
| 26.023 | Cl | 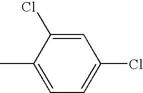 | H | —N(CH₃)₂ |
| 26.024 | Cl | 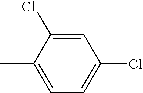 | H | ethynyl |
| 26.025 | Cl | 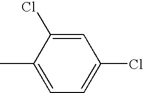 | H | cyolopropyl |
| 26.026 | Cl | 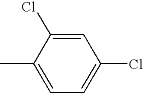 | H | 4-fluorophenyl |
| 26.027 | Cl | 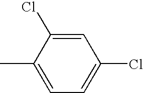 | H | phenyl |
| 26.028 | Cl | 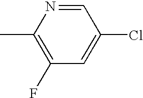 | H | —CN |
| 26.029 | Cl | 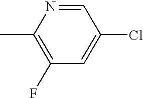 | H | —CH═CH₂ |
| 26.030 | Cl | 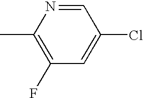 | H | —SCH₃ |
| 26.031 | Cl | 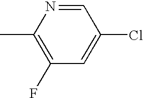 | H | —NH₂ |
| 26.032 | Cl | 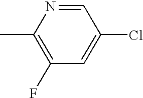 | H | —N(CH₃)₂ |
| 26.033 | Cl | 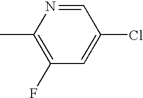 | H | ethynyl |
| 26.034 | Cl | 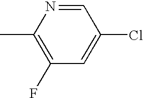 | H | cyolopropyl |
| 26.035 | Cl | 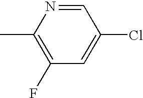 | H | 4-fluorophenyl |
| 26.036 | Cl | 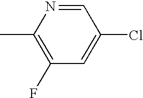 | H | phenyl |
| 26.037 | Cl | 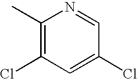 | H | —CN |
| 26.038 | Cl | 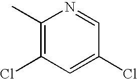 | H | —CH═CH₂ |
| 26.039 | Cl | 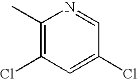 | H | —SCH₃ |
| 26.040 | Cl | 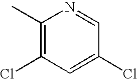 | H | —NH₂ |
| 26.041 | Cl | 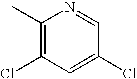 | H | —N(CH₃)₂ |
| 26.042 | Cl | 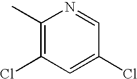 | H | ethynyl |
| 26.043 | Cl | 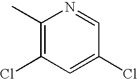 | H | cyclopropyl |
| 26.044 | Cl | 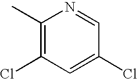 | H | 4-fluorophenyl |
| 26.045 | Cl | 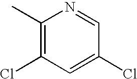 | H | phenyl |
| 26.046 | Cl | 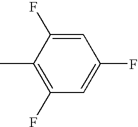 | —CN | H |
| 26.047 | Cl | 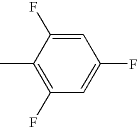 | —CH═CH₂ | H |
| 26.048 | Cl | 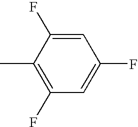 | —SCH₃ | H |
| 26.049 | Cl | 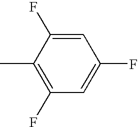 | —NH₂ | H |
| 26.050 | Cl | 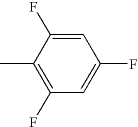 | —N(CH₃)₂ | H |
| 26.051 | Cl | 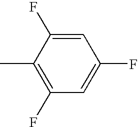 | ethynyl | H |
| 26.052 | Cl | 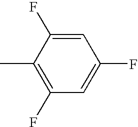 | cyclopropyl | H |
| 26.053 | Cl | 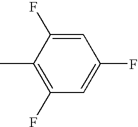 | 4-fluorophenyl | H |
| 26.054 | Cl | 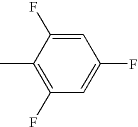 | phenyl | H |
| 26.055 | Cl | 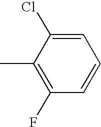 | —CN | H |
| 26.056 | Cl | 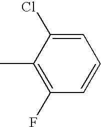 | —CH═CH₂ | H |
| 26.057 | Cl | 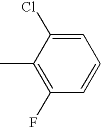 | —SCH₃ | H |
| 26.058 | Cl | 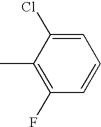 | —NH₂ | H |
| 26.059 | Cl | 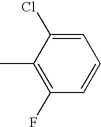 | —N(CH₃)₂ | H |
| 26.060 | Cl | 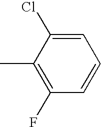 | ethynyl | H |
| 26.061 | Cl | 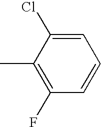 | cyclopropyl | H |
| 26.062 | Cl | 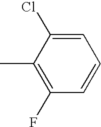 | 4-fluorophenyl | H |
| 26.063 | Cl | 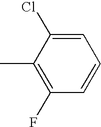 | phenyl | H |
| 26.064 | Cl | 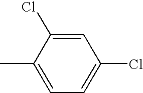 | —CN | H |
| 26.065 | Cl | 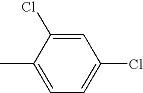 | —CH═CH₂ | H |
| 26.066 | Cl | 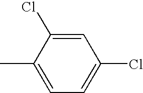 | —SCH₃ | H |
| 26.067 | Cl | 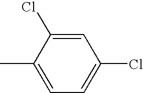 | —NH₂ | H |
| 26.068 | Cl | 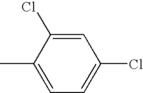 | —N(CH₃)₂ | H |
| 26.069 | Cl | 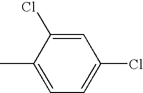 | ethynyl | H |
| 26.070 | Cl | 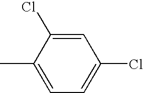 | cyclopropyl | H |
| 26.071 | Cl | 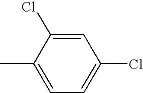 | 4-fluorophenyl | H |
| 26.072 | Cl | 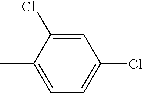 | phenyl | H |
| 26.073 | Cl | 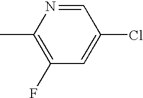 | —CN | H |
| 26.074 | Cl | 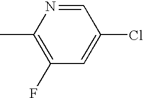 | —CH═CH₂ | H |
| 26.075 | Cl | 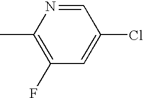 | —SCH₃ | H |
| 26.076 | Cl | 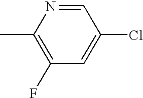 | —NH₂ | H |
| 26.077 | Cl | 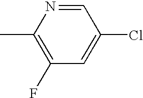 | —N(CH₃)₂ | H |
| 26.078 | Cl | 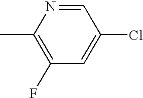 | ethynyl | H |
| 26.079 | Cl | 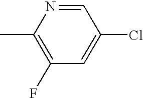 | cyclopropyl | H |
| 26.080 | Cl | 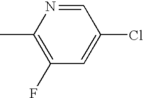 | 4-fluorophenyl | H |
| 26.081 | Cl | 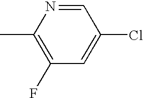 | phenyl | H |
| 26.082 | Cl | 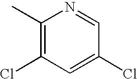 | —CN | H |
| 26.083 | Cl | 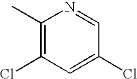 | —CH═CH₂ | H |
| 26.084 | Cl | 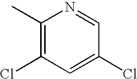 | SCH₃ | H |
| 26.085 | Cl | 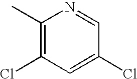 | —NH₂ | H |
| 26.086 | Cl | 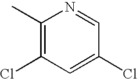 | —N(CH₃)₂ | H |
| 26.087 | Cl | 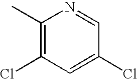 | ethynyl | H |
| 26.088 | Cl | 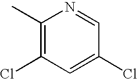 | cyclopropyl | H |
| 26.089 | Cl | 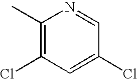 | 4-fluorophenyl | H |
| 26.090 | Cl | 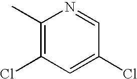 | phenyl | H |
| 26.091 | F | 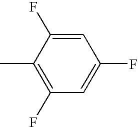 | H | —CN |
| 26.092 | F | 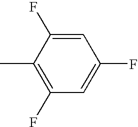 | H | —CH═CH₂ |
| 26.093 | F | 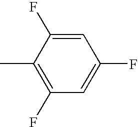 | H | —SCH₃ |
| 26.094 | F | 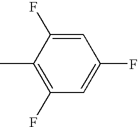 | H | —NH₂ |
| 26.095 | F | 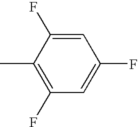 | H | —N(CH₃)₂ |
| 26.096 | F | 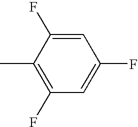 | H | ethynyl |
| 26.097 | F | 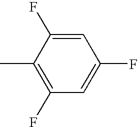 | H | cyclopropyl |
| 26.098 | F | 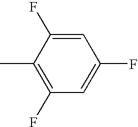 | H | 4-fluorophenyl |
| 26.099 | F | 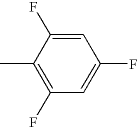 | H | phenyl |
| 26.100 | F | 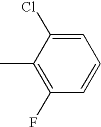 | H | —CN |
| 26.101 | F | 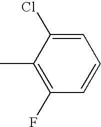 | H | —CH═CH₂ |
| 26.102 | F | 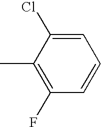 | H | —SCH₃ |
| 26.103 | F | 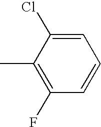 | H | —NH₂ |
| 26.104 | F | 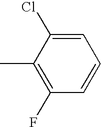 | H | —N(CH₃)₂ |
| 26.105 | F | 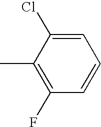 | H | ethynyl |
| 26.106 | F | 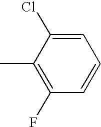 | H | cyclopropyl |
| 26.107 | F | 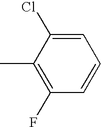 | H | 4-fluorophenyl |
| 26.108 | F | 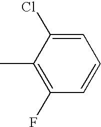 | H | phenyl |
| 26.109 | F | 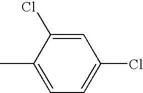 | H | —CN |
| 26.110 | F | 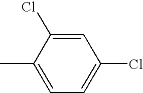 | H | —CH═CH₂ |
| 26.111 | F | 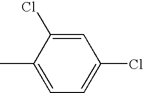 | H | —SCH₃ |
| 26.112 | F | 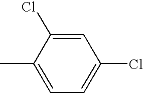 | H | —NH₂ |
| 26.113 | F | 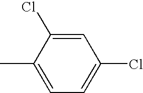 | H | —N(CH₃)₂ |
| 26.114 | F | 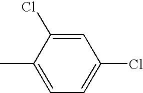 | H | ethynyl |
| 26.115 | F | 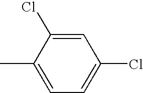 | H | cyclopropyl |
| 26.116 | F | 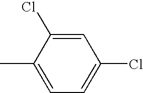 | H | 4-fluorophenyl |
| 26.117 | F | 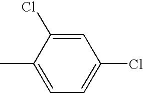 | H | phenyl |
| 26.118 | F | 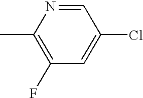 | H | —CN |
| 26.119 | F | 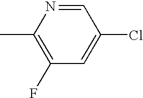 | H | —CH═CH₂ |
| 26.120 | F | 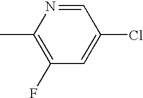 | H | —SCH₃ |
| 26.121 | F | 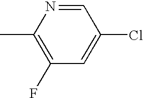 | H | —NH₂ |
| 26.122 | F | 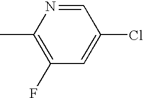 | H | —N(CH₃)₂ |
| 26.123 | F | 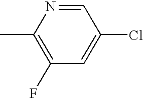 | H | ethynyl |
| 26.124 | F | 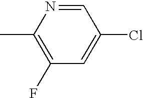 | H | cyclopropyl |
| 26.125 | F | 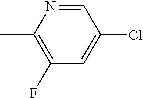 | H | 4-fluorophenyl |
| 26.126 | F | 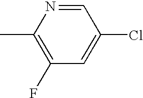 | H | phenyl |
| 26.127 | F | 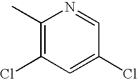 | H | —CN |
| 26.128 | F | 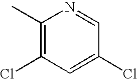 | H | —CH═CH₂ |
| 26.129 | F | 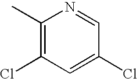 | H | —SCH₃ |
| 26.130 | F | 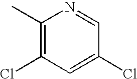 | H | —NH₂ |
| 26.131 | F | 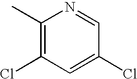 | H | —N(CH₃)₂ |
| 26.132 | F | 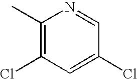 | H | ethynyl |
| 26.133 | F | 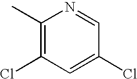 | H | cyclopropyl |
| 26.134 | F | 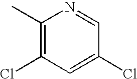 | H | 4-fluorophenyl |
| 26.135 | F | 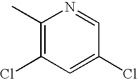 | H | phenyl |
| 26.136 | F | 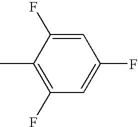 | —CN | H |
| 26.137 | F | 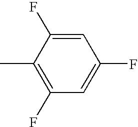 | —CH═CH₂ | H |
| 26.138 | F | 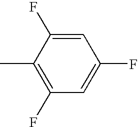 | —SCH₃ | H |
| 26.139 | F | 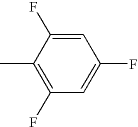 | —NH₂ | H |
| 26.140 | F | 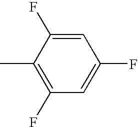 | —N(CH₃)₂ | H |
| 26.141 | F | 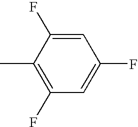 | ethynyl | H |
| 26.142 | F | 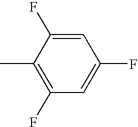 | cyclopropyl | H |
| 26.143 | F | 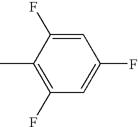 | 4-fluorophenyl | H |
| 26.144 | F | 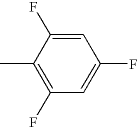 | phenyl | H |
| 26.145 | F | 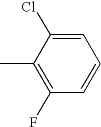 | —CN | H |
| 26.146 | F | 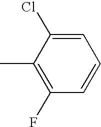 | —CH═CH₂ | H |
| 26.147 | F | 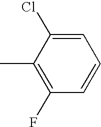 | —SCH₃ | H |
| 26.148 | F | 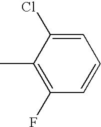 | —NH₂ | H |
| 26.149 | F | 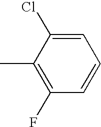 | —N(CH₃)₂ | H |
| 26.150 | F | 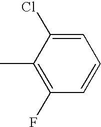 | ethynyl | H |
| 26.151 | F | 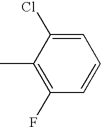 | cyclopropyl | H |
| 26.152 | F | 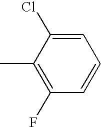 | 4-fluorophenyl | H |
| 26.153 | F | 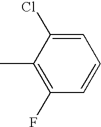 | phenyl | H |
| 26.154 | F | 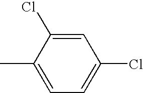 | —CN | H |
| 26.155 | F | 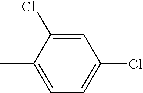 | —CH═CH₂ | H |
| 26.156 | F | 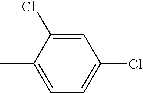 | —SCH₃ | H |
| 26.157 | F | 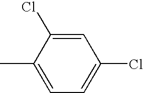 | —NH₂ | H |
| 26.158 | F | 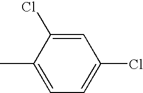 | —N(CH₃)₂ | H |
| 26.159 | F | 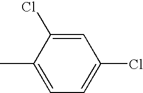 | ethynyl | H |
| 26.160 | F | 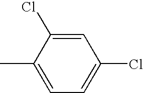 | cyclopropyl | H |
| 26.161 | F | 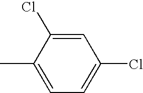 | 4-fluorophenyl | H |
| 26.162 | F | 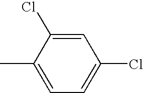 | phenyl | H |
| 26.163 | F | 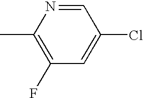 | —CN | H |
| 26.164 | F | 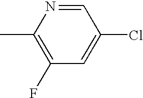 | —CH═CH₂ | H |
| 26.165 | F | 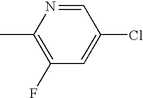 | —SCH₃ | H |
| 26.166 | F | 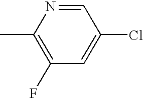 | —NH₂ | H |
| 26.167 | F | 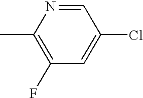 | —N(CH₃)₂ | H |
| 26.168 | F | 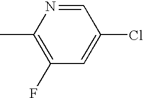 | ethynyl | H |
| 26.169 | F | 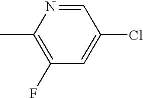 | cyclopropyl | H |
| 26.170 | F | 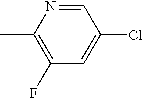 | 4-fluorophenyl | H |
| 26.171 | F | 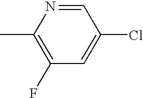 | phenyl | H |
| 26.172 | F | 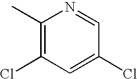 | —CN | H |
| 26.173 | F | 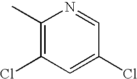 | —CH═CH₂ | H |
| 26.174 | F | 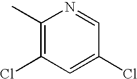 | —SCH₃ | H |
| 26.175 | F | 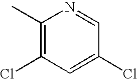 | —NH₂ | H |
| 26.176 | F | 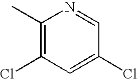 | —N(CH₃)₂ | H |
| 26.177 | F | 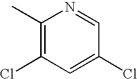 | ethynyl | H |
| 26.178 | F | 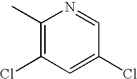 | cyclopropyl | H |
| 26.179 | F | 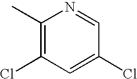 | 4-fluorophenyl | H |
| 26.180 | F | 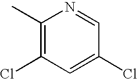 | phenyl | H |

### Table 27

Table 27 consists of 180 compounds of the general formula (1k), where R, R¹, R⁸ and R⁹ are as listed in Table 26, R² is NR³R⁴, R³ and R⁴ together are forming a piperidyl ring and the general formula (1k) is identical to general formula (1a), that is, R⁸ and R⁹ form a methyloxazole ring as depicted below.

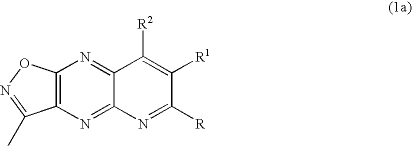

### Table 28

Table 28 consists of 180 compounds of the general formula (1k), where R, R¹, R⁸ and R⁹ are as listed in Table 26, R² is NR³R⁴, R³ and R⁴ together are forming a piperidyl ring and the general formula (1k) is identical to general formula (1b), that is, R⁸ and R⁹ form a cyclopentyl ring as depicted below.

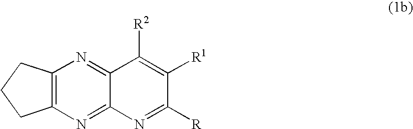

### Table 29

Table 29 consists of 180 compounds of the general formula (1k), where R, R¹, R⁸ and R⁹ are as listed in Table 26, R² is NR³R⁴, R³ and R⁴ together are forming a piperidyl ring and the general formula (1k) is identical to general formula (1c), that is, R⁸ and R⁹ form a cyclohexyl ring oxazole ring as depicted below.

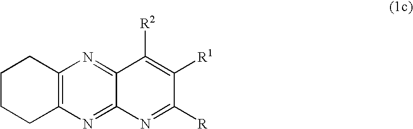

### Table 30

Table 30 consists of 180 compounds of the general formula (1k), where R, R¹, R⁸ and R⁹ are as listed in Table 26, R² is NR³R⁴, R³ and R⁴ together are forming a piperidyl ring and the general formula (1k) is identical to general formula (1d), that is, R⁸ and R⁹ form a thiazole ring as depicted below.

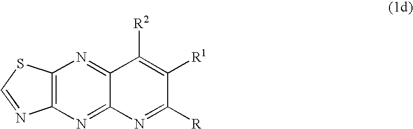

### Table 31

Table 31 consists of 180 compounds of the general formula (1k), where R, R¹, R⁸ and R⁹ are as listed in Table 26, R² is NR³R⁴, R³ and R⁴ together are forming a piperidyl ring and the general formula (1k) is identical to general formula (1e), that is, R⁸ and R⁹ form an thiazole ring as depicted below.

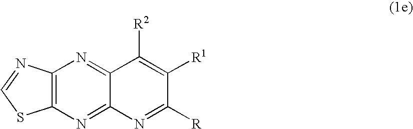

### Table 32

Table 32 consists of 180 compounds of the general formula (1k), where R, R¹, R⁸ and R⁹ are as listed in Table 26, R² is NR³R⁴, R³ and R⁴ together are forming a piperidyl ring and the general formula (1k) is identical to general formula (1f), that is, R⁸ and R⁹ form a methylpyrazole ring as depicted below.

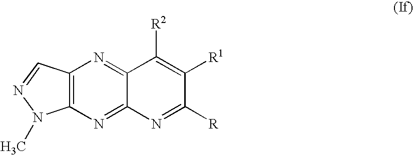

Compounds of general formula (7) and (8), which are examples of compound of general formula (1) can be made according to Scheme 1. Compounds of general formula (4) can be prepared from compounds of general formula (2) by reaction with acids of general formula (3), using standard coupling methods, for example by conversion to the acid chloride using a chlorinating agent such as thionyl chloride, followed by reaction of the resultant acid chloride optionally in the presence of a base such as triethylamine, in a suitable solvent such as dichloromethane or toluene. Compounds of general formula (5) can be prepared by treating compounds of general formula (4) with a base such as potassium carbonate, in a suitable solvent such as N,N-dimethylformamide (DMF) at between room temperature and 150° C., but preferably at 60-90° C., or a base such as sodium hexamethyldisilazide in THF at a temperature between −78° C. and room temperature, but preferably between −40° C. and 0° C. Compounds of general formula (6) can be prepared by reaction of compounds of general formula (5) with a chlorination reagent such as phosphorus oxychloride, either neat or in a suitable solvent such as toluene, at between 50 and 150° C., but preferably between 80 and 110° C., or in a microwave reactor at between 150 and 300° C., but preferably between 200 and 250° C. Compounds of formula (7) and (8) can be prepared by reaction of compounds of general formula (6) with an amine R³R⁴NH, either neat, or in a suitable solvent such as DMF, between room temperature and 150° C., but preferably between 50 and 80° C. If compounds (7) and (8) are produced as a mixture they can be separated by suitable means such as crystallisation or chromatography under normal or reverse phase conditions.

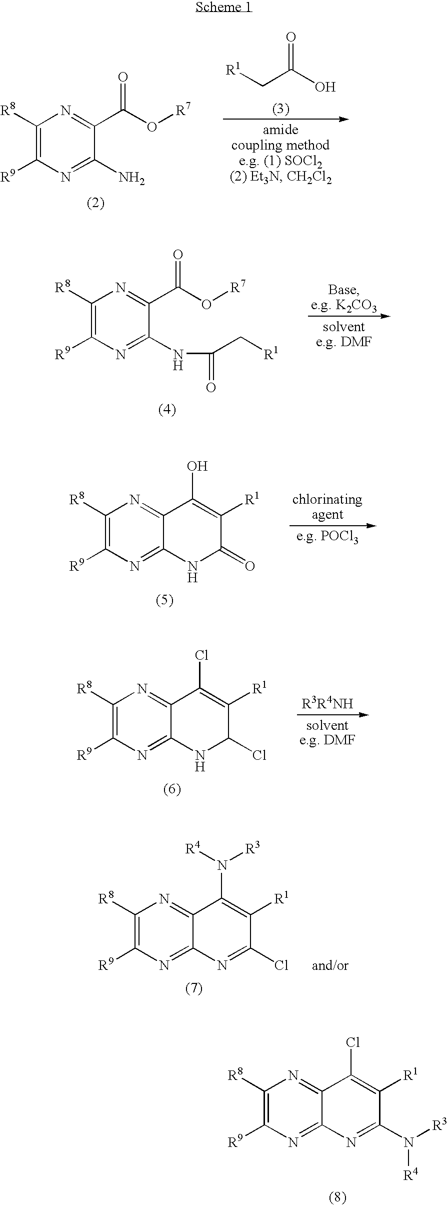

Compounds of general formula (19) and (20), which are examples of compounds of general formula (1) where R⁹ is H, can be made as shown in Schemes 2 and 3. Scheme 2 shows the preparation of compounds of general formula (15) and (16), which can then be used to prepare compounds of general formula (19) and (20) as shown in Scheme 3.

In Scheme 2 compounds of general formula (10), can be prepared from compounds of general formula (9), where Hal is a fluoro, chloro, bromo or iodo group, which are known in the literature, for example WO2001060834 for Hal is fluoro, BE623480 for Hal is chloro, W. J. Wayne et al, Journal of Organic Chemistry, 53, (9), 2052-5 (1988) for Hal is bromo, and BE6234840 for Hal is iodo, using methods A to F which are described below.

In method A, for example where R⁸ is an aryl or heteroaryl group, compounds of general formula (9) are reacted with an aryl or heteroaryl boronic acid, such as phenylboronic acid, using methods in the literature such as those by W. J. Wayne et al, Journal of Organic Chemistry, 53, (9), 2052-5 (1988), for example in the presence of a palladium catalyst such as tetrakis(triphenylphosphine)palladium, and a base such as potassium carbonate, in a suitable solvent such as toluene, at between room temperature and reflux but preferably at 50-100° C.

In Method B, to make compounds of formula (10) where R⁸ is a C₂₋₄alkenyl group, compounds of general formula (9) are reacted with a C₂₋₄alkenyl boronic acid, such as vinylboronic acid, in the presence of a palladium catalyst such as tetrakis(triphenylphosphine)palladium, and a base such as potassium carbonate, in a suitable solvent such as toluene, at between room temperature and reflux but preferably at 50-100° C.

In Method C, to make compounds of formula (10) where R⁸ is a C₂₋₄alkynyl group, compounds of general formula (9) can be reacted with a C₂₋₄alkyne, such as propyne, in the presence of a palladium catalyst such as tetrakis(triphenylphosphine)palladium and an amine such as triethylamine and a copper catalyst such as cuprous iodide.

In Method D, to make compounds of formula (10) where R⁸ is a C₁₋₄ alkoxy group, compounds of general formula (9) can be reacted with a C₁₋₄ alkanol, such as methanol, in a suitable solvent such as methanol, in the presence of a base such as potassium carbonate, or sodium methoxide.

In Method E, to make compounds of formula (10), where R⁸ is a C₁₋₄ alkylthio group, compounds of general formula (9) can be reacted with a C₁₋₄ alkanethiol, such as methanethiol, in a suitable solvent such as methanol, in the presence of a base such as potassium carbonate or sodium methoxide, as described in U.S. Pat. No. 4,481,202.

In Method F, to make compounds of formula (10) where R⁸ is a group NR¹⁰R¹¹, compounds of general formula can be reacted with an amine HNR¹⁰R¹¹, in a suitable solvent such as DMF in the presence of a base such as potassium carbonate, or an excess of the amine, as described in WO9304048.

The sequence of reactions to make compounds of formula (16) from compounds of formula (10) are exactly the same as those shown in Scheme 1 for converting compounds of formula (2) into compounds of formula (6), whereby compounds of formula (10) are converted into compounds of formula (12), which are in turn converted into compounds of formula (14), which are in turn converted into compounds of formula (16).

It should be noted that there are alternative routes from compounds of formula (9) to compounds of formula (16), whereby the conversion of the Hal group into the group of R⁸ by

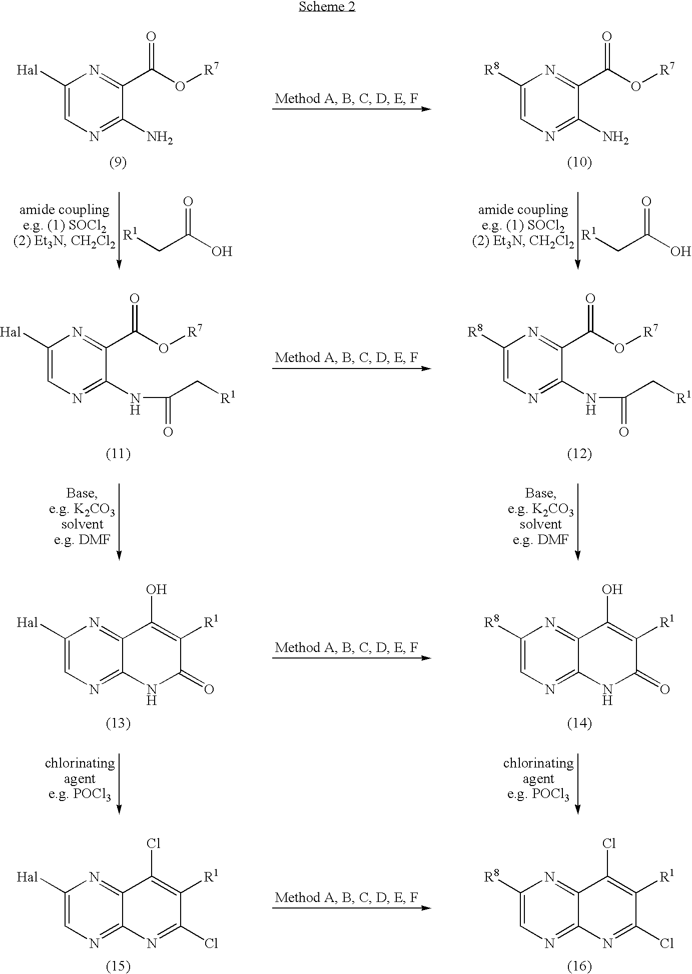

The sequence of reactions to make compounds of formula (19) and (20) from compounds of formula (15) and (16), as shown in Scheme 3, are exactly the same as those shown in Scheme 1 for converting compounds of formula (6) into compounds of formula (7) and (8), whereby compounds of formula (16) are reacted with an amine R³R⁴NH, either neat, or in a suitable solvent such as DMF, between room temperature and 150° C., but preferably between 50 and 80° C.

It should be noted that there are alternative routes, also shown in Scheme 3, whereby compounds of formula (15) are converted into compounds of formula (17) and (18), which are in turn converted into compounds of formula (19) and (20) using any of the methods A to F.

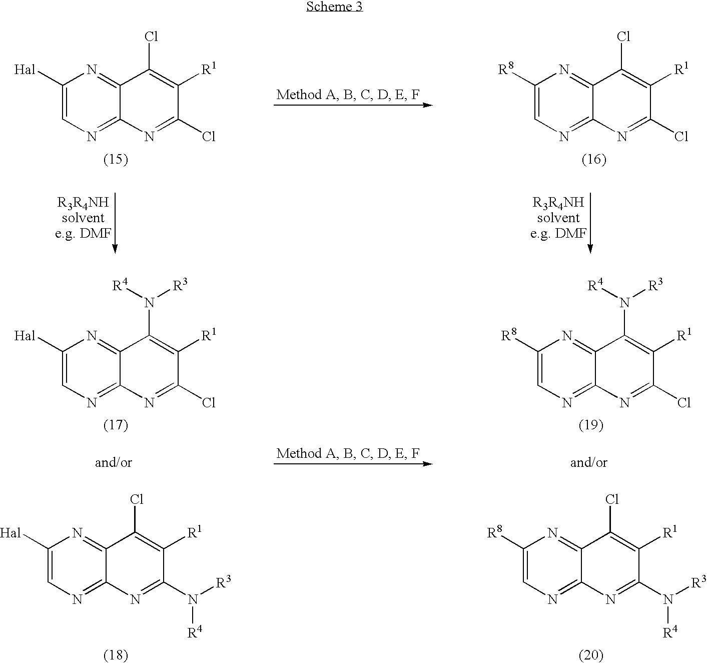

Schemes 2 and 3 show the preparation of compounds of formula (19) and (20) which are examples of compounds of general formula (1) where R⁹ is H. For the preparation of compounds of general formula (21) and (22), which are examples of compounds of general formula (1) where R⁸ is H, exactly the same sequences of reactions as in Schemes (2) and (3) can be used, except that the part structure (23) is replaced by part structure (24) throughout, and the part structure (25) is replaced by part structure (26) throughout.

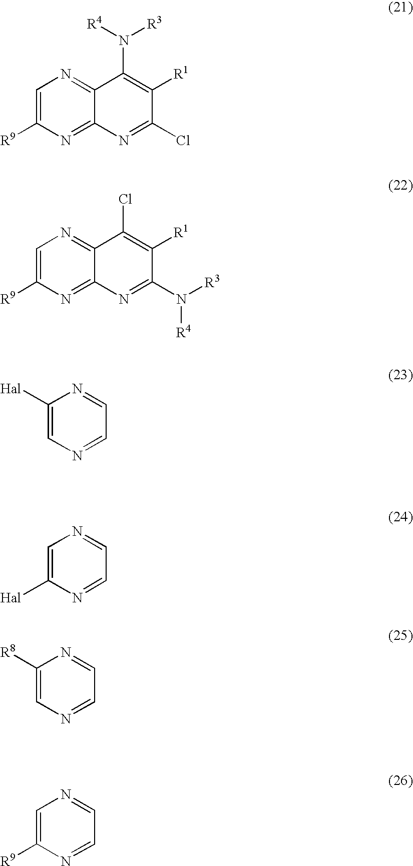

Compounds of formula (28), (29) and/or (30) can be prepared as shown in Scheme 3. Compounds of formula (27) are prepared by reaction of compounds of formula (6) with a fluoride ion source, such as potassium fluoride, in a suitable solvent such as sulpholane at a temperature between 50° C. and 200° C., preferably at 80-150° C. Compounds of formula (28), (29) and (30) can be prepared from difluoro compounds of formula (27) by reaction with an amine of formula R³R⁴NH in a suitable solvent such as DMF or CH₂Cl₂ at a temperature of 0° C.-100° C., but preferably at room temperature. Generally compounds of formula (28) are produced as a single isomer, but they may be in mixture with compounds of formula (29). Compounds of formula (30) are produced if an excess of the amine R³R⁴NH is used.

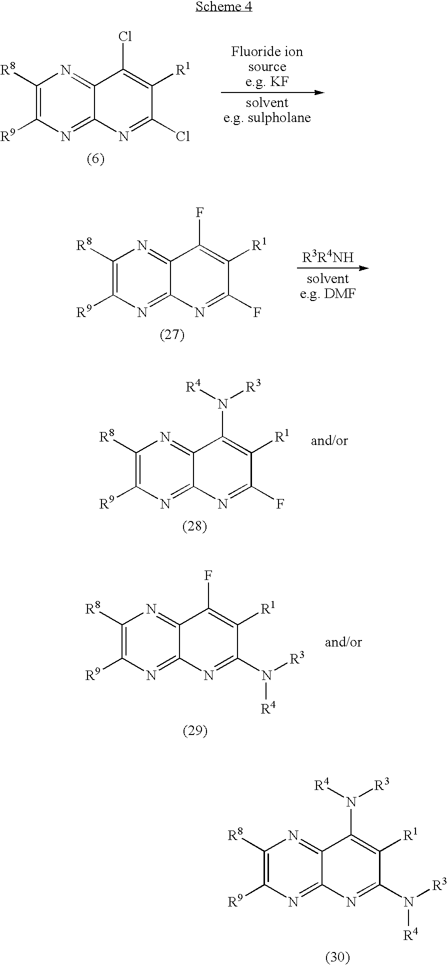

Further assistance in the preparation of the compounds of formula (1) may be derived from the following publications: Emilio, Toja, et al., J. Heterocyclic Chem., 23, 1955 (1986), H. Schafer, et. al., J. f. prakt. Chemie, 321(4), 695 (1970) and H. Bredereck et. al., Chem. Ber. 96, 1868-1872 (1993).

The intermediate chemicals having the general formulae (4), (5), (6) and (27):

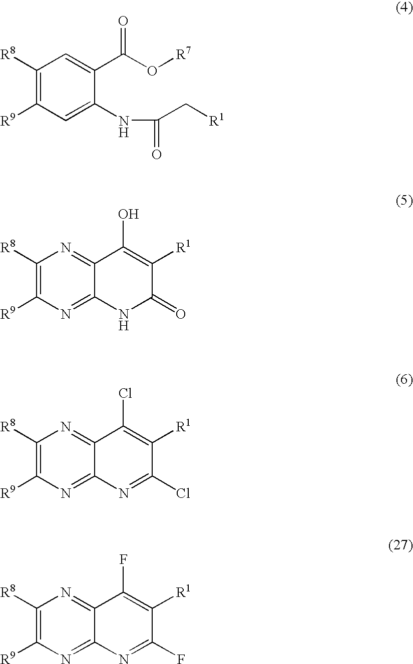

wherein R¹, R⁷, R⁸ and R⁹ are as define above, are believed to be novel compounds and form a further part of this invention.

It should be noted that the intermediate of general formula (5) may exist in the tautomeric forms (a), (b) and (c) as well as in the form shown in formula (5):

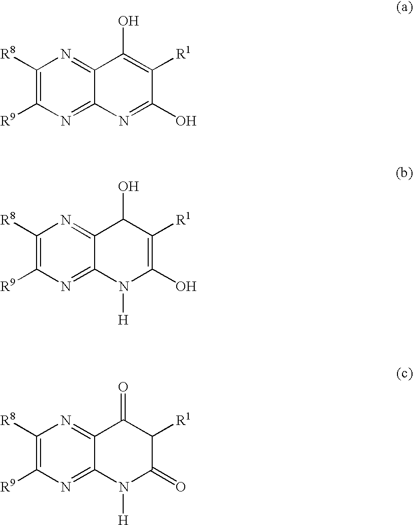

The invention as defined by the general formula (5) embraces all such tautomers.

The compounds of formula (1) are active fungicides and may be used to control one or more of the following pathogens: Pyricularia oryzae (Magnaporthe grisea) on rice and wheat and other Pyricularia spp. on other hosts; Puccinia triticina (or recondita), Puccinia strilformis and other rusts on wheat, Puccinia hordei, Puccinia struiformis and other rusts on barley, and rusts on other hosts (for example turf, rye, coffee, pears, apples, peanuts, sugar beet, vegetables and ornamental plants); Erysiphe cichoracearum on cucurbits (for example melon); Blumeria (or Erysiphe) graminis (powdery mildew) on barley, wheat, rye and turf and other powdery mildews on various hosts, such as Sphaerotheca macularis on hops, Sphaerotheca fusca (Sphaerotheca fuliginea) on cucurbits (for example cucumber), Leveillula taurica on tomatoes, aubergine and green pepper, Podosphaera leucotricha on apples and Uncinula necator on vines; Cochliobolus spp., Helminthosporium spp., Drechslera spp. (Pyrenophora spp.), Rhynchosporium spp., Mycosphaerella graminicola (Septoria tritici) and Phaeosphaeria nodorum (Stagonospora nodorum or Septoria nodorum), Pseudocercosporella herpotrichoides and Gaeumannomyces graminis on cereals (for example wheat, barley, rye), turf and other hosts; Cercospora arachidicola and Cercosporidium personatum on peanuts and other Cercospora spp. on other hosts, for example sugar beet, bananas, soya beans and rice; Botrytis cinerea (grey mould) on tomatoes, strawberries, vegetables, vines and other hosts and other Botrytis spp. on other hosts; Alternaria spp. on vegetables (for example carrots), oil-seed rape, apples, tomatoes, potatoes, cereals (for example wheat) and other hosts; Venturia spp. (including Venturia inaequalis (scab)) on apples, pears, stone fruit, tree nuts and other hosts; Cladosporium spp. on a range of hosts including cereals (for example wheat) and tomatoes; Monilinia spp. on stone fruit, tree nuts and other hosts; Didymella spp. on tomatoes, turf, wheat, cucurbits and other hosts; Phoma spp. on oil-seed rape, turf, rice, potatoes, wheat and other hosts; Aspergillus spp. and Aureobasidium spp. on wheat, lumber and other hosts; Ascochyta spp. on peas, wheat, barley and other hosts; Stemphylium spp. (Pleospora spp.) on apples, pears, onions and other hosts; summer diseases (for example bitter rot (Glomerella cingulata), black rot or frogeye leaf spot (Botryosphaeria obtusa), Brooks fruit spot (Mycosphaerella pomi), Cedar apple rust (Gymnosporangium juniperi-virginianae), sooty blotch (Gloeodes pomigena), flyspeck (Schizothyrium pomi) and white rot (Botryosphaeria dothidea)) on apples and pears; Plasmopara viticola on vines; other downy mildews, such as Bremia lactucae on lettuce, Peronospora spp. on soybeans, tobacco, onions and other hosts, Pseudoperonospora humuli on hops and Pseudoperonospora cubensis on cucurbits; Pythium spp. (including Pythium ultimum) on turf and other hosts; Phytophthora infestans on potatoes and tomatoes and other Phytophthora spp. on vegetables, strawberries, avocado, pepper, ornamentals, tobacco, cocoa and other hosts; Thanatephorus cucumeris on rice and turf and other Rhizoctonia spp. on various hosts such as wheat and barley, peanuts, vegetables, cotton and turf; Sclerotinia spp. on turf, peanuts, potatoes, oil-seed rape and other hosts; Sclerotium spp. on turf, peanuts and other hosts; Gibberella fujikuroi on rice; Colletotrichum spp. on a range of hosts including turf, coffee and vegetables; Laetisaria fuciformis on turf; Mycosphaerella spp. on bananas, peanuts, citrus, pecans, papaya and other hosts; Diaporthe spp. on citrus, soybean, melon, pears, lupin and other hosts; Elsinoe spp. on citrus, vines, olives, pecans, roses and other hosts; Verticillium spp. on a range of hosts including hops, potatoes and tomatoes; Pyrenopeziza spp. on oil-seed rape and other hosts; Oncobasidium theobromae on cocoa causing vascular streak dieback; Fusarium spp., Typhula spp., Microdochium nivale, Ustilago spp., Urocystis spp., Tilletia spp. and Claviceps purpurea on a variety of hosts but particularly wheat, barley, turf and maize; Ramularia spp. on sugar beet, barley and other hosts; post-harvest diseases particularly of fruit (for example Penicillium digitatum, Penicillium italicum and Trichoderma viride on oranges, Colletotrichum musae and Gloeosporium musarum on bananas and Botrytis cinerea on grapes); other pathogens on vines, notably Eutypa lata, Guignardia bidwellii, Phellinus igniarus, Phomopsis viticola, Pseudopeziza tracheiphila and Stereum hirsutum; other pathogens on trees (for example Lophodermium seditiosum) or lumber, notably Cephaloascus fragrans, Ceratocystis spp., Ophiostoma piceae, Penicillium spp., Trichoderma pseudokoningii, Trichoderma viride, Trichoderma harzianum, Aspergillus niger, Leptographium lindbergi and Aureobasidium pullulans; and fungal vectors of viral diseases (for example Polymyxa graminis on cereals as the vector of barley yellow mosaic virus (BYMV) and Polymyxa betae on sugar beet as the vector of rhizomania).

A compound of formula (1) may move acropetally, basipetally or locally in plant tissue to be active against one or more fungi. Moreover, a compound of formula (1) may be volatile enough to be active in the vapour phase against one or more fungi on the plant.

The invention therefore provides a method of combating or controlling phytopathogenic fungi which comprises applying a fungicidally effective amount of a compound of formula (1), or a composition containing a compound of formula (1), to a plant, to a seed of a plant, to the locus of the plant or seed or to soil or any other plant growth medium, e.g. nutrient solution.

The term “plant” as used herein includes seedlings, bushes and trees. Furthermore, the fungicidal method of the invention includes protectant, curative, systemic, eradicant and antisporulant treatments.

The compounds of formula (1) are preferably used for agricultural, horticultural and turfgrass purposes in the form of a composition.

In order to apply a compound of formula (1) to a plant, to a seed of a plant, to the locus of the plant or seed or to soil or any other growth medium, a compound of formula (1) is usually formulated into a composition which includes, in addition to the compound of formula (1), a suitable inert diluent or carrier and, optionally, a surface active agent (SFA). SFAs are chemicals that are able to modify the properties of an interface (for example, liquid/solid, liquid/air or liquid/liquid interfaces) by lowering the interfacial tension and thereby leading to changes in other properties (for example dispersion, emulsification and wetting). It is preferred that all compositions (both solid and liquid formulations) comprise, by weight, 0.0001 to 95%, more preferably 1 to 85%, for example 5 to 60%, of a compound of formula (1). The composition is generally used for the control of fungi such that a compound of formula (1) is applied at a rate of from 0.1 g to 10 kg per hectare, preferably from 1 g to 6 kg per hectare, more preferably from 1 g to 1 kg per hectare.

When used in a seed dressing, a compound of formula (1) is used at a rate of 0.0001 g to 10 g (for example 0.001 g or 0.05 g), preferably 0.005 g to 10 g, more preferably 0.005 g to 4 g, per kilogram of seed.

In another aspect the present invention provides a fungicidal composition comprising a fungicidally effective amount of a compound of formula (1) and a suitable carrier or diluent therefor.

In a still further aspect the invention provides a method of combating and controlling fungi at a locus, which comprises treating the fungi, or the locus of the fungi with a fungicidally effective amount of a composition comprising a compound of formula (1).

The compositions can be chosen from a number of formulation types, including dustable powders (DP), soluble powders (SP), water soluble granules (SG), water dispersible granules (WG), wettable powders (WP), granules (GR) (slow or fast release), soluble concentrates (SL), oil miscible liquids (OL), ultra low volume liquids (UL), emulsifiable concentrates (EC), dispersible concentrates (DC), emulsions (both oil in water (EW) and water in oil (EO)), micro-emulsions (ME), suspension concentrates (SC), aerosols, fogging/smoke formulations, capsule suspensions (CS) and seed treatment formulations. The formulation type chosen in any instance will depend upon the particular purpose envisaged and the physical, chemical and biological properties of the compound of formula (1).

Dustable powders (DP) may be prepared by mixing a compound of formula (1) with one or more solid diluents (for example natural clays, kaolin, pyrophyllite, bentonite, alumina, montmorillonite, kieselguhr, chalk, diatomaceous earths, calcium phosphates, calcium and magnesium carbonates, sulphur, lime, flours, talc and other organic and inorganic solid carriers) and mechanically grinding the mixture to a fine powder.

Soluble powders (SP) may be prepared by mixing a compound of formula (1) with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of formula (1) with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of formula (1) and one or more powdered solid diluents or carriers, or from pre-formed blank granules by absorbing a compound of formula (1) (or a solution thereof, in a suitable agent) in a porous granular material (such as pumice, attapulgite clays, fuller's earth, kieselguhr, diatomaceous earths or ground corn cobs) or by adsorbing a compound of formula (1) (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of formula (1) in water or an organic solvent, such as a ketone, alcohol or glycol ether. These solutions may contain a surface active agent (for example to improve water dilution or prevent crystallisation in a spray tank).

Emulsifiable concentrates (EC) or oil-in-water emulsions (EW) may be prepared by dissolving a compound of formula (1) in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone), alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment. Preparation of an EW involves obtaining a compound of formula (1) either as a liquid (if it is not a liquid at room temperature, it may be melted at a reasonable temperature, typically below 70° C.) or in solution (by dissolving it in an appropriate solvent) and then emulsifying the resultant liquid or solution into water containing one or more SFAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents that have a low solubility in water.

Microemulsions (ME) may be prepared by mixing water with a blend of one or more solvents with one or more SFAs, to produce spontaneously a thermodynamically stable isotropic liquid formulation. A compound of formula (1) is present initially in either the water or the solvent/SFA blend. Suitable solvents for use in MEs include those hereinbefore described for use in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of formula (1). SCs may be prepared by ball or bead milling the solid compound of formula (1) in a suitable medium, optionally with one or more dispersing agents, to produce a fine particle suspension of the compound. One or more wetting agents may be included in the composition and a suspending agent may be included to reduce the rate at which the particles settle. Alternatively, a compound of formula (1) may be dry milled and added to water, containing agents hereinbefore described, to produce the desired end product.

Aerosol formulations comprise a compound of formula (1) and a suitable propellant (for example n-butane). A compound of formula (1) may also be dissolved or dispersed in a suitable medium (for example water or a water miscible liquid, such as n-propanol) to provide compositions for use in non-pressurised, hand-actuated spray pumps.

A compound of formula (1) may be mixed in the dry state with a pyrotechnic mixture to form a composition suitable for generating, in an enclosed space, a smoke containing the compound.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of formula (1) and, optionally, a carrier or diluent therefor. The polymeric shell may be produced by either an interfacial polycondensation reaction or by a coacervation procedure. The compositions may provide for controlled release of the compound of formula (1) and they may be used for seed treatment. A compound of formula (1) may also be formulated in a biodegradable polymeric matrix to provide a slow, controlled release of the compound.

A composition may include one or more additives to improve the biological performance of the composition (for example by improving wetting, retention or distribution on surfaces; resistance to rain on treated surfaces; or uptake or mobility of a compound of formula (1)). Such additives include surface active agents, spray additives based on oils, for example certain mineral oils or natural plant oils (such as soy bean and rape seed oil), and blends of these with other bio-enhancing adjuvants (ingredients which may aid or modify the action of a compound of formula (1)).

A compound of formula (1) may also be formulated for use as a seed treatment, for example as a powder composition, including a powder for dry seed treatment (DS), a water soluble powder (SS) or a water dispersible powder for slurry treatment (WS), or as a liquid composition, including a flowable concentrate (FS), a solution (LS) or a capsule suspension (CS). The preparations of DS, SS, WS, FS and LS compositions are very similar to those of, respectively, DP, SP, WP, SC and DC compositions described above. Compositions for treating seed may include an agent for assisting the adhesion of the composition to the seed (for example a mineral oil or a film-forming barrier).

Wetting agents, dispersing agents and emulsifying agents may be SFAs of the cationic, anionic, amphoteric or non-ionic type.

Suitable SFAs of the cationic type include quaternary ammonium compounds (for example cetyltrimethyl ammonium bromide), imidazolines and amine salts.

Suitable anionic SFAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefine sulphonates, taurates and lignosulphonates.

Suitable SFAs of the amphoteric type include betaines, propionates and glycinates.

Suitable SFAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); and lecithins.

Suitable suspending agents include hydrophilic colloids (such as polysaccharides, polyvinylpyrrolidone or sodium carboxymethylcellulose) and swelling clays (such as bentonite or attapulgite).

A compound of formula (1) may be applied by any of the known means of applying fungicidal compounds. For example, it may be applied, formulated or unformulated, to any part of the plant, including the foliage, stems, branches or roots, to the seed before it is planted or to other media in which plants are growing or are to be planted (such as soil surrounding the roots, the soil generally, paddy water or hydroponic culture systems), directly or it may be sprayed on, dusted on, applied by dipping, applied as a cream or paste formulation, applied as a vapour or applied through distribution or incorporation of a composition (such as a granular composition or a composition packed in a water-soluble bag) in soil or an aqueous environment.

A compound of formula (1) may also be injected into plants or sprayed onto vegetation using electrodynamic spraying techniques or other low volume methods, or applied by land or aerial irrigation systems.

Compositions for use as aqueous preparations (aqueous solutions or dispersions) are generally supplied in the form of a concentrate containing a high proportion of the active ingredient, the concentrate being added to water before use. These concentrates, which may include DCs, SCs, ECs, EWs, MEs SGs, SPs, WPs, WGs and CSs, are often required to withstand storage for prolonged periods and, after such storage, to be capable of addition to water to form aqueous preparations which remain homogeneous for a sufficient time to enable them to be applied by conventional spray equipment. Such aqueous preparations may contain varying amounts of a compound of formula (1) (for example 0.0001 to 10%, by weight) depending upon the purpose for which they are to be used.

A compound of formula (1) may be used in mixtures with fertilisers (for example nitrogen-, potassium- or phosphorus-containing fertilisers). Suitable formulation types include granules of fertiliser. The mixtures suitably contain up to 25% by weight of the compound of formula (1).

The invention therefore also provides a fertiliser composition comprising a fertiliser and a compound of formula (1).

The compositions of this invention may contain other compounds having biological activity, for example micronutrients or compounds having similar or complementary fungicidal activity or which possess plant growth regulating, herbicidal, insecticidal, nematicidal or acaricidal activity.

By including another fungicide, the resulting composition may have a broader spectrum of activity or a greater level of intrinsic activity than the compound of formula (1) alone. Further the other fungicide may have a synergistic effect on the fungicidal activity of the compound of formula (1).

The compound of formula (1) may be the sole active ingredient of the composition or it may be admixed with one or more additional active ingredients such as a pesticide, fungicide, synergist, herbicide or plant growth regulator where appropriate. An additional active ingredient may: provide a composition having a broader spectrum of activity or increased persistence at a locus; synergise the activity or complement the activity (for example by increasing the speed of effect or overcoming repellency) of the compound of formula (1); or help to overcome or prevent the development of resistance to individual components. The particular additional active ingredient will depend upon the intended utility of the composition.

Examples of fungicidal compounds which may be included in the composition of the invention are AC 382042 (N-(1-cyano-1,2-dimethylpropyl)-2-(2,4-dichlorophenoxy) propionamide), acibenzolar-S-methyl, alanycarb, aldimorph, anilazine, azaconazole, azafenidin, azoxystrobin, benalaxyl, benomyl, benthiavalicarb, biloxazol, bitertanol, blasticidin S, boscalid (new name for nicobifen), bromuconazole, bupirimate, captafol, captan, carbendazim, carbendazim chlorhydrate, carboxin, carpropamid, carvone, CGA 41396, CGA 41397, chinomethionate, chlorbenzthiazone, chlorothalonil, chlorozolinate, clozylacon, copper containing compounds such as copper oxychloride, copper oxyquinolate, copper sulphate, copper tallate, and Bordeaux mixture, cyamidazosulfamid, cyazofamid (IKF-916), cyflufenamid, cymoxanil, cyproconazole, cyprodinil, debacarb, di-2-pyridyl disulphide 1,1′-dioxide, dichlofluanid, diclocymet, diclomezine, dicloran, diethofencarb, difenoconazole, difenzoquat, diflumetorim, O,O-di-isopropyl-5-benzyl thiophosphate, dimefluazole, dimetconazole, dimethirimol, dimethomorph, dimoxystrobin, diniconazole, dinocap, dithianon, dodecyl dimethyl ammonium chloride, dodemorph, dodine, doguadine, edifenphos, epoxiconazole, ethaboxam, ethirimol, ethyl (Z)-N-benzyl-N([methyl(methyl-thioethylideneaminooxycarbonyl)amino]thio)-β-alaninate, etridiazole, famoxadone, fenamidone, fenarimol, fenbuconazole, fenfuram, fenhexamid, fenoxanil (AC 382042), fenpiclonil, fenpropidin, fenpropimorph, fentin acetate, fentin hydroxide, ferbam, ferimzone, fluazinam, fludioxonil, flumetover, flumorph, fluoroimide, fluoxastrobin, fluquinconazole, flusilazole, flusulfamide, flutolanil, flutriafol, folpet, fosetyl-aluminium, fuberidazole, furalaxyl, furametpyr, guazatine, hexaconazole, hydroxyisoxazole, hymexazole, imazalil, imibenconazole, iminoctadine, iminoctadine triacetate, ipconazole, iprobenfos, iprodione, iprovalicarb, isopropanyl butyl carbamate, isoprothiolane, kasugamycin, kresoxim-methyl, LY186054, LY211795, LY 248908, mancozeb, maneb, mefenoxam, mepanipyrim, mepronil, metalaxyl, metalaxyl M, metconazole, metiram, metiram-zinc, metominostrobin, metrafenone, MON65500 (N-allyl-4,5-dimethyl-2-trimethylsilylthiophene-3-carboxamide), myclobutanil, NTN0301, neoasozin, nickel dimethyldithiocarbamate, nitrothale-isopropyl, nuarimol, ofurace, organomercury compounds, orysastrobin, oxadixyl, oxasulfuron, oxolinic acid, oxpoconazole, oxycarboxin, pefurazoate, penconazole, pencycuron, phenazin oxide, phosphorus acids, phthalide, picoxystrobin, polyoxin D, polyram, probenazole, prochloraz, procymidone, propamocarb, propamocarb hydrochloride, propiconazole, propineb, propionic acid, proquinazid, prothioconazole, pyraclostrobin, pyrazophos, pyrifenox, pyrimethanil, pyroquilon, pyroxyfur, pyrrolnitrin, quaternary ammonium compounds, quinomethionate, quinoxyfen, quintozene, silthiofam (MON 65500), S-imazalil, simeconazole, sipconazole, sodium pentachlorophenate, spiroxamine, streptomycin, sulphur, tebuconazole, tecloftalam, tecnazene, tetraconazole, thiabendazole, thifluzamide, 2-(thiocyanomethylthio)-benzothiazole, thiophanate-methyl, thiram, tiadinil, timibenconazole, tololofos-methyl, tolylfluanid, triadimefon, triadimenol, triazbutil, triazoxide, tricyclazole, tridemorph, trifloxystrobin, triflumizole, triforine, triticonazole, validamycin A, vapam, vinclozolin, XRD-563, zineb, ziram, zoxamide and compounds of the formulae:

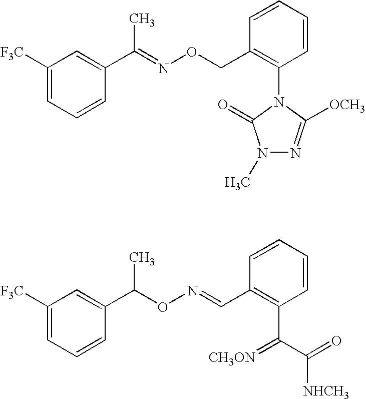

The compounds of formula (1) may be mixed with soil, peat or other rooting media for the protection of plants against seed-borne, soil-borne or foliar fungal diseases.

Some mixtures may comprise active ingredients, which have significantly different physical, chemical or biological properties such that they do not easily lend themselves to the same conventional formulation type. In these circumstances other formulation types may be prepared. For example, where one active ingredient is a water insoluble solid and the other a water insoluble liquid, it may nevertheless be possible to disperse each active ingredient in the same continuous aqueous phase by dispersing the solid active ingredient as a suspension (using a preparation analogous to that of an SC) but dispersing the liquid active ingredient as an emulsion (using a preparation analogous to that of an EW). The resultant composition is a suspoemulsion (SE) formulation.

The invention is illustrated by the following Examples in which the following abbreviations are used:

| | |
|---|---|
| ml = millilitres | f = fine |
| g = grammes | THF = tetrahydrofuran |
| ppm = parts per million | DCM = dichloromethane |
| s = singlet | DMF = N,N-dimethylformamide |
| d = doublet | DMSO = dimethylsulphoxide |
| t = triplet | DMAP = 4-dimethylaminopyridine |
| q = quartet | NMR = nuclear magnetic resonance |
| m = multiplet | HPLC = high performance liquid |
| b = broad | chromatography |

### EXAMPLE 1

This Example illustrates the preparation of (1,2-Dimethyl-propyl)-[6-fluoro-2-(4-fluorophenyl)-7-(2,4,6-trifluorophenyl)-pyrido[2,3-b]pyrazin-8-yl]-amine (Compound No. ?, Table 1).

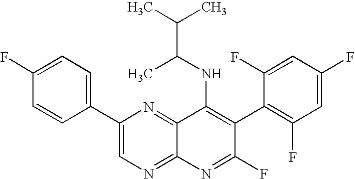

### Step 1

The preparation of 3-amino-6-(4-fluorophenyl)-pyrazine-2-carboxylic acid methyl ester:

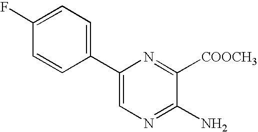

Palladium acetate (0.145 g) and [1,1′-bis(diphenylphosphino)ferrocene]dichloropalladium complex with DCM (0.702 g) were dissolved in DMF (65 ml) and heated to 50° C. for 15 minutes. After cooling to room temperature 3-amino-6-bromo-pyrazine-2-carboxylic acid methyl ester (5.0 g), 4-fluoro-phenyl boronic acid (3.82 g) and triethylamine (4.5 ml) were added and the mixture heated to 90° C. for 20 hours with stirring. The solvent was evaporated and the residue dissolved in DCM. The organic phase was washed with dilute ammonium hydroxide solution and water. After drying with sodium sulphate and filtration the solvent was evaporated and the residue purified by column chromatography on silica gel eluting with hexane:ethyl acetate, 3:1 to give 3-amino-6-(4-fluorophenyl)-pyrazine-2-carboxylic acid methyl ester (0.670 g).

¹H NMR (CDCl₃) δ ppm: 3.92 (s, 3H), 6.4 (bs, 1H), 7.1 (m, 2H), 7.8 (m, 2H), 8.5 (s, 1H).

### Step 2

The preparation of 6-(4-fluorophenyl)-3-[2-(2,4,6-trifluorophenyl)-acetylamino]-pyrazine-2-carboxylic acid methyl ester:

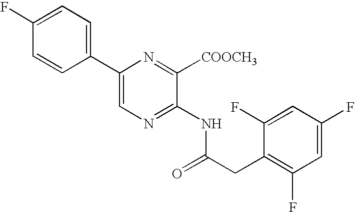

The product from Step 1 (010 g), 2,4,6-trifluorophenylacetyl chloride (0.091 g) were mixed in toluene (4.5 ml) and heated to 120° C. overnight. The cooled reaction mixture was poured into water and the product extracted with ethyl acetate. The organic layer was dried over sodium sulphate and filtered and the solvent evaporated. The residue was purified by column chromatography on silica gel eluting with hexane:ethyl acetate, 9:1 to give of 6-(4-fluorophenyl)-3-[2-(2,4,6-trifluorophenyl)-acetylamino]-pyrazine-2-carboxylic acid methyl ester (0.045 g).

¹H NMR (CDCl₃) δ ppm: 3.92 (s, H), 6.67 (m, 2H), 7.72 (m, 2H), 7.92 (m, 2H), 8.87 (s, 1H), 10.6 (s, 1H).

### Step 3

The preparation of 2-(4-fluorophenyl)-8-hydroxy-7-(2,4,6-trifluorophenyl)-5H-pyrido[2,3-b]pyrazin-6-one:

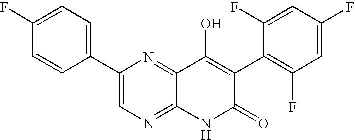

The product from Step 2 (0.5 g) and potassium carbonate (0.33 g) were added to DMF (50 ml) and stirred at 60° C. for 1 hour. The reaction mixture was poured into ice and water, acidified to pH 3 and filtered. The solid was dried to give 2-(4-fluoro-phenyl)-8-hydroxy-7-(2,4,6-trifluorophenyl)-5H-pyrido[2,3-b]pyrazin-6-one (0.405 g), m.p. 285-288.5° C. In analogy to this compound, 2-phenyl-8-hydroxy-7-(2,4,6-trifluorophenyl)-5H-pyrido[2,3-b]pyrazin-6-one was prepared.

NMR (DMSO d6): 7.27 m (2H); 7.50 m (3H); 8.50 m (2H); 9.42 s (1H); 11.35 s (1H); 12.12s (1H).

### Step 4

The preparation of 6,8-dichloro-2-(4-fluorophenyl)-7-(2,4,6-trifluorophenyl)-pyrido[2,3-b]pyrazine:

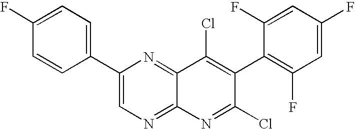

The product from Step 3 (0.330 g) and DMF (0.125 g) were mixed in 1,2-dichloroethane (2 ml) and heated to 80° C. Phosphorous oxychloride (0.522 g) was added slowly, and heating continued for 2 hours. The reaction mixture was slowly poured into ice and water. The product was extracted with diethyl ether, which after washing with brine, drying and evaporation of the solvent, was purified by flash column chromatography on silica gel, eluting with hexane:ethyl acetate, 9:1, to give 6,8-dichloro-2-(4-fluorophenyl)-7-(2,4,6-trifluorophenyl)-pyrido[2,3-b]pyrazine, (0.260 g).

¹H NMR (CDCl₃) δ ppm: 6.85 (m, 2H), 7.25 (m, 2H), 8.27 (m, 2H), 9.6 (s, 1H).

In analogy to this compound, 6,8-dichloro-2-phenyl-7-(2,4,6-trifluorophenyl)-pyrido[2,3-b]pyrazine has been prepared.

NMR (CDCl₃): 6.85 m (2H); 7.6 m (3H); 8.27 m (2H); 9.62 s (1H).

### Step 5

The preparation of 6,8-difluoro-2-(4-fluorophenyl)-7-(2,4,6-trifluorophenyl)-pyrido[2,3-b]pyrazine:

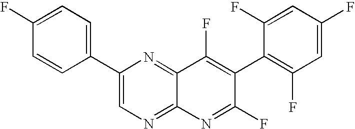

The product from Step 4 (0.050 g) and dry potassium fluoride (0.021 g) were added to dry sulpholane (3.3 ml) and heated to 130° C. with stirring for 5 hours. After cooling, water was added and the product extracted with diethyl ether. The organic phase was washed with brine, dried over sodium sulphate, filtered and the solvent evaporated. The residue was purified by flash column chromatography over silica gel eluting with hexane:ethyl acetate, 9:1 to give 6,8-difluoro-2-(4-fluorophenyl)-7-(2,4,6-trifluorophenyl)-pyrido[2,3-b]pyrazine (0.046 g).

¹H NMR (CDCl₃) δ ppm: 6.87 (m, 2H), 7.25 (m, 2H), 8.22 (m, 2H), 9.57 (s, 1H).

In analogy to this compound, 6,8-difluoro-2-phenyl-7-(2,4,6-trifluorophenyl)-pyrido[2,3-b]pyrazine was prepared.

NMR (CDCl₃): 6.85 m (2H); 7.57 m (3H); 8.2 m (2H); 9.6 s (1H).

### Step 6

The preparation of (1,2-dimethylpropyl)-[6-fluoro-2-(4-fluorophenyl)-7-(2,4,6-trifluorophenyl)-pyrido[2,3-b]pyrazin-8-yl]amine, Compound 18.038:

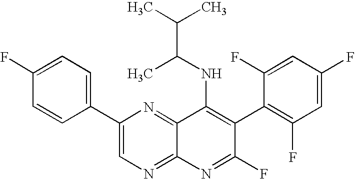

The product from Step 5 (0.020 g), 1,2-dimethylpropylamine (0.009 g), a catalytic amount of DMAP and dry DMF (4 ml) were mixed and stirred for 1 hour at room temperature. Water was added to the reaction mixture and the product extracted with diethyl ether. After drying, the solvent was evaporated and the residue purified by flash column chromatography over aluminum oxide, eluting with hexane:ethyl acetate, 4:1 to give the title compound (0.011 g).

¹H NMR (CDCl₃) δ ppm: 0.75 (d, 6H), 0.97 (d, 3H), 1.6 (m, 1H), 3.07 (m, 1H), 6.77 (m, 2H), 7.17 (m, 2H), 8.02 (m, 2H), 9.4 (s, 1H).

TABLE 33

| Compound | | NMR data (ppm, in CDCl₃, unless otherwise |
|---|---|---|
| No. | Compound Structure | stated), Mpt. or mass spec. M+ |
| 18.037 | 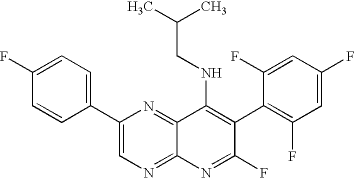 | M + 1, 442 |
| | 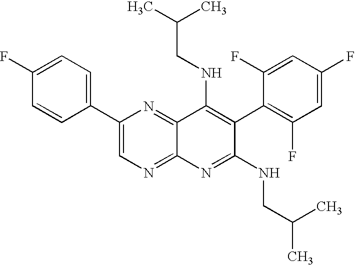 | 0.75 (d, 6 H), 0.80 (d. 6 H), 1.65 (m, 1 H), 1.82(m, 1 H), 2.5 (m, 2 H), 3.3 (m, 2 H), 4.3 (m, 1 H),6.75 (m, 1 H), 6.85 (m, 2 H), 7.12 (m, 2 H), 7.9(m, 2 H), 9.07 (s, 1 H) |
| 18.036 | 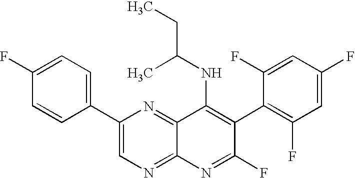 | 0.72 (t, 3 H), 1.02 (d, 3 H), 1.42 (m, 2 H), 3.15(m, 1 H), 6.77 (m, 1 H), 7.0 (d, 1 H), 7.2 (m, 3 H),8.0 (m, 2 H), 9.4 (s, 1 H). |
| 19.036 | 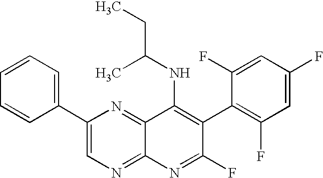 | 0.72 (t, 3 H), 1.05 (d, 3 H), 1.4 (m, 2 H), 3.15(m, 1 H), 6.77 (m, 1 H), 7.05 (d, 1 H), 7.5 (m, 3 H),8.07 (m, 2 H), 9.45 (s, 1 H). |
| 19.037 | 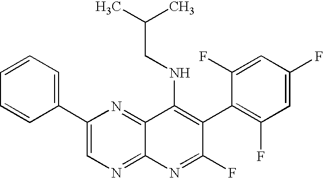 | 0.5 (d, 6 H), 1.72 (d, 1 H), 2.75 (m, 2 H), 6.75(m, 2 H), 7.32 (d, 1 H), 7.5 (m, 3 H), 8.02 (m, 2 H),9.47 (s, 1 H). |
| 19.038 | 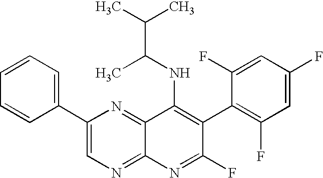 | 0.77 (d, 6 H), 0.98 (m, 3 H), 1.62 (m, 1 H), 3.1(m, 1 H), 6.77 (m, 2 H), 7.5 (m, 3 H), 8.05 (m, 2 H),9.45 (s, 1 H). |

### EXAMPLE 2

This Example illustrates the fungicidal properties of the compounds of the general formula (1).

Compounds were tested in a leaf disk assay, with methods described below. Test compounds were dissolved in DMSO, and diluted into water to 20 ppm. Pyricularia orzyae (rice blast): Conidia of the fungus from cryogenic storage were directly mixed into nutrient broth (PDB potato dextrose broth). After placing a (DMSO) solution of the test compounds into a microtiter plate (96-well format) the nutrient broth containing the fungal spores was added. The test plates were incubated at 24 C and the inhibition of growth was determined photometrically after 72 hours.

The following compounds gave greater than 60% control of disease:

Pyricularia orzyae: 18.037

1. The compound of the general formula (1): 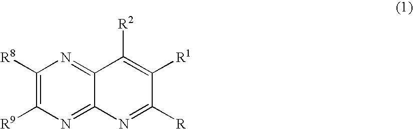
R is H, halo, C₁₋₄ alkyl, C₁₋₄alkoxy, halo(C₁₋₄)alkyl, cyano or NR³R⁴;
R¹ is aryl, aryloxy, arylthio, heteroaryl, heteroaryloxy, heteroarylthio, aryl(C₁₋₄)alkyl, aryl(C₁₋₄)alkoxy, heteroaryl(C₁₋₄)alkyl, heteroaryl(C₁₋₄)alkoxy;
R² is halo or NR³R⁴;
R³ and R⁴ are independently H, C₁₋₈ alkyl, C₂₋₈ alkenyl, C₂₋₈ alkynyl, aryl, aryl(C₁₋₈)alkyl, C₃₋₈ cycloalkyl, C₃₋₈cycloalkyl(C₁₋₆)alkyl, heteroaryl, heteroaryl(C₁₋₈)alkyl, NR⁵R⁶, provided that not both R³ and R⁴ are H or NR⁵R⁶, or
R³ and R⁴ together form a C₃₋₇ alkylene or C₃₋₇ alkenylene chain optionally substituted with one or more C₁₋₄ alkyl or C₁₋₄ alkoxy groups, or,
together with the nitrogen atom to which they are attached, R³ and R⁴ form a morpholine, thiomorpholine, thiomorpholine S-oxide, or thiomorpholine S-dioxide ring, or a piperazine or piperazine N—(C₁₋₄)alkyl (especially N-methyl) ring or a pyrrolidine ring;
R⁵ and R⁶ are independently H, C₁₋₈ alkyl, C₂₋₈ alkenyl, C₂₋₈ alkynyl, aryl, aryl(C₁₋₈)alkyl, C₃₋₈ cycloalkyl, C₃₋₈ cycloalkyl(C₁₋₆)alkyl, heteroaryl or heteroaryl(C₁₋₈)alkyl;
R⁸ and R⁹ can be H, halo, C₁₋₄alkyl, C₁₋₄alkoxy, halo(C₁₋₄)alkyl, CN, C₁₋₄alkylthio, C₁₋₄alkylsulphinyl, C₁₋₄alkylsulphonyl, aryl, heteroaryl, halo(C₁₋₆)alkoxy, halo(C₁₋₄)alkylthio, C₂₋₄alkenyl, C₂₋₆alkynyl, C₃₋₆cycloalkyl, C₁₋₆alkoxycarbonyl, R³R⁴NCO, NR¹⁰R¹¹, or R³ and R⁹ can be joined to form a saturated or unsaturated 5-7 membered carbocyclic or heterocyclic ring, optionally substituted with one or two substituents R¹² and where the heterocyclic ring can contain from one to three heteroatoms chosen from NR¹³, O or S;
R¹⁰ and R¹¹ are independently H, C₁₋₈ alkyl, C₂₋₈alkenyl, C₂₋₈ alkynyl, C₁₋₄ alkoxy, aryl, aryl(C₁₋₈)alkyl, C₃₋₈cycloalkyl, C₃₋₈cycloalkyl(C₁₋₆)alkyl, heteroaryl, heteroaryl(C₁₋₈)alkyl, NR⁵R⁶, provided that not both R⁹ and R¹⁰ are NR⁵R⁶ or C₁₋₄alkoxy;
R¹² is H, halo, C₁₋₄alkyl, C₁₋₄alkoxy, halo(C₁₋₄)alkyl, CN, C₁₋₄alkylthio, C₁₋₄alkylsulphinyl, C₁₋₄alkylsulphonyl, aryl, heteroaryl, halo(C₁₋₆)alkoxy, halo(C₁₋₄)alkylthio, C₂₋₄alkenyl, C₂₋₆alkynyl, C₃₋₆cycloalkyl, NR¹⁰R¹¹, aryl, heteroaryl, halo(C₁₋₆)alkoxy, halo(C₁₋₄)alkylthio, C₂₋₄alkenyl, C₂₋₆alkynyl, C₃₋₆cycloalkyl, C₁₋₆alkoxycarbonyl or R³R⁴NCO;
R¹³ is H, C₁₋₄alkyl or halo(C₁₋₄)alkyl;
any of the foregoing alkyl, alkenyl, alkynyl or cycloalkyl groups or moieties being optionally substituted with halogen, cyano, C₁₋₆ alkoxy, C₁₋₆ alkylcarbonyl, C₁₋₆ alkoxycarbonyl, C₁₋₆ haloalkoxy, C₁₋₆ alkylthio, tri(C₁₋₄)alkylsilyl, C₁₋₆ alkylamino or C₁₋₆ dialkylamino,
any of the foregoing morpholine, thiomorpholine, piperidine, piperazine and pyrrolidine rings being optionally substituted with C₁₋₄ alkyl (especially methyl), and
any of the foregoing aryl or heteroaryl groups or moieties being optionally substituted with one or more substituents selected from halo, hydroxy, mercapto, C₁₋₆ alkyl, C₂₋₆ alkenyl, C₂₋₆ alkynyl, C₁₋₆ alkoxy, C₂₋₆ alkenyloxy, C₂₋₆ alkynyloxy, halo(C₁₋₆)alkyl, halo(C₁₋₆)alkoxy, C₁₋₆ alkylthio, halo(C₁₋₆)-alkylthio, hydroxy(C₁₋₆)alkyl, C₁₋₄ alkoxy(C₁₋₆)alkyl, C₃₋₆ cycloalkyl, C₃₋₆ cycloalkyl(C₁₋₄)alkyl, aryl, heteroaryl, aryloxy, heteroaryloxy, aryl(C₁₋₄)alkoxy, heteroaryl(C₁₋₄)alkoxy, aroyloxy, arylthio, arylsulphinyl, arylsulphonyl, heteroarylthio, heteroarylsulphinyl, heteroarylsulphonyl, arylalkenyl, arylalkynyl, heteroarylalkenyl, heteroarylalkynyl, aryl(C₁₋₄)alkyl, heteroaryl(C₁₋₄)alkyl, cyano, isocyano, thiocyanato, isothiocyanato, nitro, —NR¹⁰³R¹⁰⁴, —NHCOR¹⁰³, —NHCONR¹⁰³R¹⁰⁴, —CONR¹⁰³R¹⁰⁴, —SO₂R¹⁰³, —OSO₂R¹⁰³, —COR¹⁰³, —CR¹⁰³═NR¹⁰⁴ or —N═CR¹⁰³R¹⁰⁴ in which R¹⁰³ and R¹⁰⁴ are independently hydrogen, C₁₋₄ alkyl, halo(C₁₋₄)alkyl, C₁₋₄ alkoxy, halo(C₁₋₄)alkoxy, C₁₋₄ alkylthio, C₃₋₆ cycloalkyl, C₃₋₆ cycloalkyl(C₁₋₄)alkyl, phenyl or benzyl, with any of the forgoing aryl or heteroaryl substituents being optionally substituted with halo, C₁₋₆ alkyl, C₂₋₆ alkenyl, C₂₋₆ alkynyl, C₁₋₆ alkoxy, C₂₋₆ alkenyloxy, C₂₋₆ alkynyloxy, halo(C₁₋₆)alkyl, halo(C₁₋₆)alkoxy, C₁₋₆ alkylthio, halo(C₁₋₆)alkylthio, C₁₋₄ alkoxy(C₁₋₆)alkyl, C₃₋₆ cycloalkyl, cyano or nitro, provided that not both of R³ and R⁹ are independently hydrogen, halogen, C₁₋₄alkyl, C₁₋₄alkoxy, haloC₁₋₄alkyl.

2. A compound according to claim 1 wherein R⁸ or R⁹ is equal to H.

3. A compound according to claim 1 wherein R² is NR³R⁴.

4. A compound according to claim 1 wherein R is halo.

5. A process for preparing a compound of the general formula (1) according to claim 1 wherein one of R and R² is chloro or fluoro and the other is NR³R⁴ and R¹, R³, R⁴, R⁸ and R⁹ are as defined in claim 1, which comprises reacting an amine of the general formula NR³R⁴ with a compound of the general formula (6) or (27):

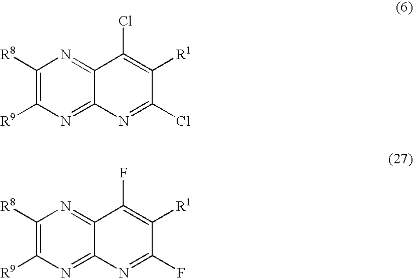

6. The intermediate chemicals having the general formulae (4), (5), (6) and (27): 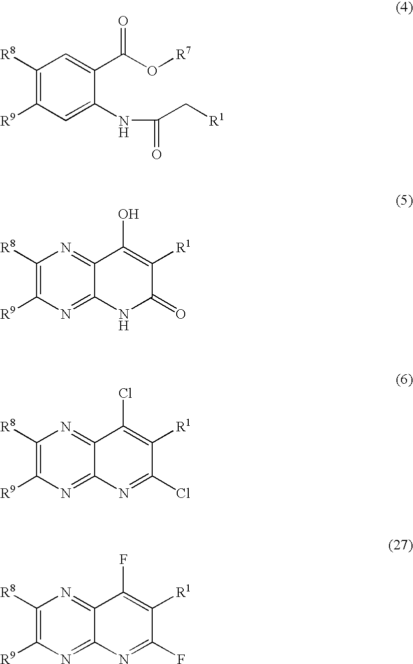
wherein R¹, R⁸ and R⁹ are as defined in claim 1 and R⁷ is C₁₋₄ alkyl.

7. A plant fungicidal composition comprising a fungicidally effective amount of a compound as defined in claim 1.

8. A method of combating or controlling phytopathogenic fungi which comprises applying to a plant, to a seed of a plant, to the locus of the plant or seed or to soil or to any other plant growth medium, a fungicidally effective amount of a compound according to claim 1 or a composition including said compound.
